SEQUENCE LISTING (84 sequences)

SEQ ID NO: 1 - protein, 724 aa, Artificial Sequence
  PCY1 enzyme (S. vaccaria)
MATSGFSKPLHYPPVRRDETVVDDYFGVKVADPYRWLEDPNSEETKEFVDNQEKLANSVLEECELIDKFKQKIIDFVNFPRCGVPFRRANKYFHFYNSGLQAQNVFQMQDDLDGKPEVLYDPNLREGGRSGLSLYSVSEDAKYFAFGIHSGLTEWVTIKILKTEDRSYLPDTLEWVKFSPAIWTHDNKGFFYCPYPPLKEGEDHMTRSAVNQEARYHFLGTDQSEDILLWRDLENPAHHLKCQITDDGKYFLLYILDGCDDANKVYCLDLTKLPNGLESFRGREDSAPFMKLIDSFDASYTAIANDGSVFTFQTNKDAPRKKLVRVDLNNPSVWTDLVPESKKDLLESAHAVNENQLILRYLSDVKHVLEIRDLESGALQHRLPIDIGSVDGITARRRDSVVFFKFTSILTPGIVYQCDLKNDPTQLKIFRESVVPDFDRSEFEVKQVFVPSKDGTKIPIFIAARKGISLDGSHPCEMHGYGGFGINMMPTFSASRIVFLKHLGGVFCLANIRGGGEYGEEWHKAGFRDKKQNVFDDFISAAEYLISSGYTKARRVAIEGGSNGGLLVAACINQRPDLFGCAEANCGVMDMLRFHKFTLGYLWTGDYGCSDKEEEFKWLIKYSPIHNVRRPWEQPGNEETQYPATMILTADHDDRVVPLHSFKLLATMQHVLCTSLEDSPQKNPIIARIQRKAAHYGRATMTQIAEVADRYGFMAKALEAPWID

SEQ ID NO: 2 - protein, 730 aa, Artificial Sequence
  GmPOPB
MSSVTWAPGNYPSTRRSDHVDTYQSASKGEVPVPDPYQWLEESTDEVDKWTTAQADLAQSYLDQNADIQKLAEKFRASRNYAKFSAPTLLDDGHWYWFYNRGLQSQSVLYRSKEPALPDFSKGDDNVGDVFFDPNVLAADGSAGMVLCKFSPDGKFFAYAVSHLGGDYSTIYVRSTSSPLSQASVAQGVDGRLSDEVKWFKFSTIIWTKDSKGFLYQRYPARERHEGTRSDRNAMMCYHKVGTTQEEDIIVYQDNEHPEWIYGADTSEDGKYLYLYQFKDTSKKNLLWVAELDEDGVKSGIHWRKVVNEYAADYNIITNHGSLVYIKTNLNAPQYKVITIDLSKDEPEIRDFIPEEKDAKLAQVNCANEEYFVAIYKRNVKDEIYLYSKAGVQLTRLAPDFVGAASIANRQKQTHFFLTLSGFNTPGTIARYDFTAPETQRFSILRTTKVNELDPDDFESTQVWYESKDGTKIPMFIVRHKSTKFDGTAAAIQYGYGGFATSADPFFSPIILTFLQTYGAIFAVPSIRGGGEFGEEWHKGGRRETKVNTFDDFIAAAQFLVKNKYAAPGKVAINGASNGGLLVMGSIVRAPEGTFGAAVPEGGVADLLKFHKFTGGQAWISEYGNPSIPEEFDYIYPLSPVHNVRTDKVMPATLITVNIGDGRVVPMHSFKFIATLQHNVPQNPHPLLIKIDKSWLGHGMGKPTDKNVKDAADKWGFIARALGLELKTVE

SEQ ID NO: 3 - protein, 8 aa, Artificial Sequence
  ribosomal peptide
  MISC_FEATURE (7)..(7): Xaa at position 7 = N or D
IWGIGCXP

SEQ ID NO: 4 - protein, 12 aa, Artificial Sequence
  truncated presegetalin F1 substrate
FSASYSSKPIQT

SEQ ID NO: 5 - protein, 12 aa, Artificial Sequence
  truncated presegetalin F1 substrate
FSASYSSKPIQD

SEQ ID NO: 6 - protein, 7 aa, Artificial Sequence
  catalyst peptide
RNASAPV

SEQ ID NO: 7 - protein, 7 aa, Artificial Sequence
  catalyst peptide
DNASAPV

SEQ ID NO: 8 - protein, 8 aa, Artificial Sequence
  PCY1
VTACITWP

SEQ ID NO: 9 - protein, 12 aa, Artificial Sequence
  PCY1 processed peptide
FSASYSSKPFQA

SEQ ID NO: 10 - protein, 12 aa, Artificial Sequence
  PCY1 processed peptide
FSASYSSKPIQT

SEQ ID NO: 11 - protein, 8 aa, Artificial Sequence
  catalytic peptide
RGNASAPV

SEQ ID NO: 12 - protein, 6 aa, Artificial Sequence
  catalytic peptide
RASAPV

SEQ ID NO: 13 - protein, 9 aa, Artificial Sequence
  catalytic peptide
RGGNASAPV

SEQ ID NO: 14 - protein, 8 aa, Artificial Sequence
  catalytic peptide
RANASAPV

SEQ ID NO: 15 - protein, 8 aa, Artificial Sequence
  catalytic peptide
RAANSAPV

SEQ ID NO: 16 - protein, 9 aa, Artificial Sequence
  catalytic peptide
RGANASAPV

SEQ ID NO: 17 - protein, 6 aa, Artificial Sequence
  PCY1 macrocyclase cyclisation tag
IQTQVS

SEQ ID NO: 18 - protein, 5 aa, Artificial Sequence
  PCY1 macrocyclase cyclisation tag
IQTQV

SEQ ID NO: 19 - protein, 4 aa, Artificial Sequence
  PCY1 macrocyclase cyclisation tag
IQTQ

SEQ ID NO: 20 - protein, 3 aa, Artificial Sequence
  PCY1 macrocyclase cyclisation tag
IQT

SEQ ID NO: 21 - protein, 3 aa, Artificial Sequence
  PCY1 macrocyclase cyclisation tag
IQD

SEQ ID NO: 22 - protein, 2 aa, Artificial Sequence
  PCY1 macrocyclase cyclisation tag
IQ

SEQ ID NO: 23 - protein, 6 aa, Artificial Sequence
  PCY1 macrocyclase cyclisation tag
AKDAEN

SEQ ID NO: 24 - protein, 5 aa, Artificial Sequence
  PCY1 macrocyclase cyclisation tag
AKDAE

SEQ ID NO: 25 - protein, 4 aa, Artificial Sequence
  PCY1 macrocyclase cyclisation tag
AKDA

SEQ ID NO: 26 - protein, 3 aa, Artificial Sequence
  PCY1 macrocyclase cyclisation tag
AKD

SEQ ID NO: 27 - protein, 6 aa, Artificial Sequence
  PCY1 macrocyclase cyclisation tag
FQAKDV

SEQ ID NO: 28 - protein, 5 aa, Artificial Sequence
  PCY1 macrocyclase cyclisation tag
FQAKD

SEQ ID NO: 29 - protein, 4 aa, Artificial Sequence
  PCY1 macrocyclase cyclisation tag
FQAK

SEQ ID NO: 30 - protein, 3 aa, Artificial Sequence
  PCY1 macrocyclase cyclisation tag
FQA

SEQ ID NO: 31 - protein, 2 aa, Artificial Sequence
  PCY1 macrocyclase cyclisation tag
FQ

SEQ ID NO: 32 - protein, 4 aa, Artificial Sequence
  PCY1 macrocyclase percursor peptide
  MISC_FEATURE (1)..(1): Xaa is (X1...Xm) where X1 to Xm are independently any chemical group, preferably any amino acid, and m is 5 to 10 residues
XIQT

SEQ ID NO: 33 - protein, 4 aa, Artificial Sequence
  PCY1 macrocyclase precursor peptide
  MISC_FEATURE (1)..(1): Xaa is (X1...Xm) where X1 to Xm are independently any chemical group, preferably any amino acid, and m is 5 to 10 residues
XIQD

SEQ ID NO: 34 - protein, 4 aa, Artificial Sequence
  PCY1 macrocyclase precursor peptide
  MISC_FEATURE (1)..(1): Xaa is (X1...Xm) where X1 to Xm are independently any chemical group, preferably any amino acid, and m is 5 to 10 residues
XAKD

SEQ ID NO: 35 - protein, 4 aa, Artificial Sequence
  PCY1 macrocyclase precursor peptide
  MISC_FEATURE (1)..(1): Xaa is (X1...Xm) where X1 to Xm are independently any chemical group, preferably any amino acid, and m is 5 to 10 residues
XFQA

SEQ ID NO: 36 - protein, 5 aa, Artificial Sequence
  PCY1 macrocyclase cyclisation tag
AYDGE

SEQ ID NO: 37 - protein, 4 aa, Artificial Sequence
  PCY1 macrocyclase cyclisation tag
AYDG

SEQ ID NO: 38 - protein, 5 aa, Artificial Sequence
  GmPOPB macrocyclase cyclisation tag
WTAEH

SEQ ID NO: 39 - protein, 6 aa, Artificial Sequence
  GmPOPB macrocyclase cyclisation tag
WTAEHV

SEQ ID NO: 40 - protein, 6 aa, Artificial Sequence
  precursor peptide
  MISC_FEATURE (1)..(1): Xaa is (X1...Xm) where X1 to Xm are independently any chemical group, preferably any amino acid, and m is 5 to 9 residues
XWTAEH

SEQ ID NO: 41 - protein, 7 aa, Artificial Sequence
  precursor peptide
  MISC_FEATURE (1)..(1): Xaa is (X1...Xm) where X1 to Xm are independently any chemical group, preferably any amino acid, and m is 5 to 9 residues
XWTAEHV

SEQ ID NO: 42 - protein, 7 aa, Artificial Sequence
  PCY1 macrocyclase catalytic peptide
  MISC_FEATURE (1)..(1): Xaa = R, D or E
  MISC_FEATURE (5)..(5): Xaa = A or S
XNASXPV

SEQ ID NO: 43 - protein, 8 aa, Artificial Sequence
  PCY1 macrocyclase catalytic peptide
  MISC_FEATURE (1)..(1): Xaa = R, D or E
  MISC_FEATURE (6)..(6): Xaa = A  or S
XGNASXPV

SEQ ID NO: 44 - protein, 6 aa, Artificial Sequence
  PCY1 macrocyclase catalytic peptide
  MISC_FEATURE (1)..(1): Xaa = R, D or E
  MISC_FEATURE (4)..(4): Xaa = A or S
XASXPV

SEQ ID NO: 45 - protein, 9 aa, Artificial Sequence
  PCY1 macrocyclase catalytic peptide
  MISC_FEATURE (1)..(1): Xaa = R, D or E
  MISC_FEATURE (7)..(7): Xaa = A or S
XGGNASXPV

SEQ ID NO: 46 - protein, 8 aa, Artificial Sequence
  PCY1 macrocyclase catalytic peptide
  MISC_FEATURE (1)..(1): Xaa = R, D or E
  MISC_FEATURE (6)..(6): Xaa = A or S
XANASXPV

SEQ ID NO: 47 - protein, 7 aa, Artificial Sequence
  GmPOPB macrocyclase catalytic peptide
ASGNDIC

SEQ ID NO: 48 - protein, 10 aa, Artificial Sequence
  purification tag
MRGSHHHHHH

SEQ ID NO: 49 - protein, 8 aa, Artificial Sequence
  purification tag
DYKDDDDK

SEQ ID NO: 50 - protein, 15 aa, Artificial Sequence
  purification tag
KETAAAKFERQHMDS

SEQ ID NO: 51 - protein, 8 aa, Artificial Sequence
  Strept-tag II
WSHPQFEK

SEQ ID NO: 52 - protein, 10 aa, Artificial Sequence
  c-myc
EQKLISEEDL

SEQ ID NO: 53 - protein, 12 aa, Artificial Sequence
  Curz tag 09
MKAEFRRQESDR

SEQ ID NO: 54 - protein, 12 aa, Artificial Sequence
  Cruz tag 22
MRDALDRLDRLA

SEQ ID NO: 55 - protein, 9 aa, Artificial Sequence
  catalytic peptide
FSASYSSKP

SEQ ID NO: 56 - protein, 10 aa, Artificial Sequence
  peptide
  MISC_FEATURE (3)..(3): Xaa = 8Aoc
  misc_feature (4)..(4): Xaa can be any naturally occurring amino acid
FSAXSKPIQT

SEQ ID NO: 57 - protein, 10 aa, Artificial Sequence
  peptide
  MISC_FEATURE (5)..(5): Xaa = 8Aoc
VGAGXFPIQT

SEQ ID NO: 58 - protein, 7 aa, Artificial Sequence
  C-terminal peptide
NASASPV

SEQ ID NO: 59 - protein, 6 aa, Artificial Sequence
  peptide
NASAPV

SEQ ID NO: 60 - protein, 7 aa, Artificial Sequence
  peptide
  MISC_FEATURE (5)..(5): Xaa = 8Aoc
VGAGXFP

SEQ ID NO: 61 - protein, 7 aa, Artificial Sequence
  substrate
IWGIGCN

SEQ ID NO: 62 - protein, 9 aa, Artificial Sequence
  9mer core
IWGIGCANP

SEQ ID NO: 63 - protein, 17 aa, Artificial Sequence
  recognition sequence
WTAEHVDQTLASGNDIC

SEQ ID NO: 64 - protein, 12 aa, Artificial Sequence
  truncated recognition sequence
VDQTLASGNDIC

SEQ ID NO: 65 - protein, 9 aa, Artificial Sequence
  truncated recognition sequence
TLASGNDIC

SEQ ID NO: 66 - protein, 10 aa, Artificial Sequence
  leader peptdie
MFDTNATRLP

SEQ ID NO: 67 - protein, 13 aa, Artificial Sequence
  presegetalinB1
FQAKDVENASAPV

SEQ ID NO: 68 - protein, 25 aa, Artificial Sequence
  PresegF1
FSASYSSKPIQTQVSNGMDNASAPV

SEQ ID NO: 69 - protein, 18 aa, Artificial Sequence
  PresegD1
GLSFAFPAKDAENASSPV

SEQ ID NO: 70 - protein, 19 aa, Artificial Sequence
  PresegA1
GVPVWAFQAKDVENASAPV

SEQ ID NO: 71 - protein, 18 aa, Artificial Sequence
  PresegB1
GVAWAFQAKDVENASAPV

SEQ ID NO: 72 - protein, 15 aa, Artificial Sequence
  PresegB1 variant
GVAWAFQAKDVENAS

SEQ ID NO: 73 - protein, 35 aa, Artificial Sequence
  35mer substrate peptide
MFDTNATRLPIWGIGCNPWTAEHVDQTLASGNDIC

SEQ ID NO: 74 - protein, 18 aa, Artificial Sequence
  PresegB1
GVAWAFQAKDAENASAPV

SEQ ID NO: 75 - protein, 11 aa, Artificial Sequence
  C-terminal cyclisation tag
VTACITWPFQA

SEQ ID NO: 76 - protein, 11 aa, Artificial Sequence
  C-terminal cyclisation tag
VTACITWPIQT

SEQ ID NO: 77 - protein, 13 aa, Artificial Sequence
  C-terminal cyclisation tag
VTACITWPAYDGE

SEQ ID NO: 78 - protein, 11 aa, Artificial Sequence
  C-terminal cyclisation tag
VTACITWPFQA

SEQ ID NO: 79 - protein, 11 aa, Artificial Sequence
  C-terminal cyclisation tag
VTACITWPIQT

SEQ ID NO: 80 - protein, 13 aa, Artificial Sequence
  C-terminal cyclisation tag
VTACITWPAYDGE

SEQ ID NO: 81 - protein, 10 aa, Artificial Sequence
  macrocyclic products molecules
FSASAYSSKP

SEQ ID NO: 82 - protein, 11 aa, Artificial Sequence
  macrocyclic products molecules
  MISC_FEATURE (3)..(3): Xaa = N-Me
FSXASAYSSKP

SEQ ID NO: 83 - protein, 8 aa, Artificial Sequence
  macrocyclic products molecules
  MISC_FEATURE (4)..(4): Xaa = (C)het
VTAXITWP

SEQ ID NO: 84 - protein, 7 aa, Artificial Sequence
  macrocyclic products molecules
  MISC_FEATURE (4)..(4): Xaa = 8Aoc
FSAXSKP

(19) United States
(12) Patent Application Publication
(10) Pub. No.: US 2020/0291444 A1
(43) Pub. Date: Sep. 17, 2020

(54) Macrocyclisation Tags

(71) Applicant: Oxford University Innovation LTD, Oxford Oxfordshire (GB)

(72) Inventors: Clarissa Melo Czekster, North Haugh, St. Andrews (GB); Hannes Ludewig, North Haugh, St. Andrews (GB); James Henderson Naismith, Oxford Oxfordshire (GB)

(21) Appl. No.: 16/644,885

(22) PCT Filed: Sep. 7, 2018
(86) PCT No.: PCT/EP2018/074194

§ 371 Date: Mar. 5, 2020

(30) Foreign Application Priority Data
Sep. 7, 2017 (GB) 1714372.8

(51) Int. Cl. C12P 21/02; C07K 1/107

(52) U.S. Cl. CPC C12P 21/02; C07K 1/1075

(57) ABSTRACT

This invention relates to the biosynthetic production of macrocyclic molecules from linear precursor peptides that contain highly truncated C terminal recognition sequences of 10 or fewer residues using prolyl oligopeptidase (POP) macrocyclases. This may be useful for example in the biosynthetic production of macrocyclic molecules. Methods and kits for the production of macrocyclic molecules, as well as libraries and methods of screening, are provided.

### FIELD

The present invention relates to the use of macrocyclases for the in vitro production of macrocyclic molecules.

### BACKGROUND

The class of natural products represented by ribosomally synthesized and post-translationally modified peptides (RiPPs) are of particular interest at the moment as novel therapeutics.¹ Several macrocyclic compounds including cyclic RiPPs are orally active despite the fact that they are in disagreement with Lipinskis' Rule of Five². This behaviour has led to the coining of the term ‘beyond rule of five’ to describe peptide macrocycles.³ The activity and utility of macrocyclic peptides arises from their increased stability (both chemically and to protease degradation), rigidity and hydrophobicity when compared to their linear peptide counterparts.⁴ Yet, like linear peptides, macrocyclic peptides can encode complex structural and chemical information and thus may be particularly suitable to tackle difficult targets such as protein-protein interactions¹ which are traditionally challenging to target using small molecules.¹ The de novo chemical synthesis of macrocyclic peptides is well known but has some disadvantages such as the fact that it usually requires multiple steps and the conditions necessary for the ring closing reaction need to be optimized for each⁴ variant.⁵⁻⁶ Moreover, when the active macrocycle is highly modified, as are many cyanobactins, a particular class of RiPPs, their de novo chemical synthesis is not considered practical.⁵ Biotechnology provides an alternative approach utilizing enzymes entirely in vivo or in vitro. The enzymes involved in RiPPs biosynthesis are particularly attractive as they are able to carry out highly specific modifications while operating with a very broad substrate range that is not limited to amino acids but includes many other chemical entities. This allows enzyme activities from different pathways and organisms to be combined.⁷

Cyanobactins possess a wide range of desirable and valuable bioactivities including anti-cancer (cytostatic and cytotoxic), antifouling, immunomodulating and antineoplastic.⁸ An understanding of the cyanobactin biosynthetic machinery has underpinned the production of a wide range of novel cyanobactins in vivo⁹⁻¹⁰ and in vitro.¹¹⁻¹⁴ Whilst in vivo approaches—sometimes termed ‘cell factory’—hold the advantage of simplicity, in vitro technologies have the unique capacity of harnessing the power and diversity of chemical synthesis. This means that not only multiple different non-natural amino acids can easily be accommodated, but hybrid molecules (peptide and non-peptide) that are impossible to be produced in vivo can be generated.¹³

To date, the majority of reports of in vitro macrocyclisation have used the macrocyclase domain from the patellamide pathway (PatGmac). Although extremely promiscuous, the enzyme has extremely slow catalytic rates in vitro, a major drawback that limits its application at large scale or in highly parallel processes.¹⁵ A fast and promiscuous macrocyclase enzyme is thus highly desirable. Of the macrocyclases known to date, PatGmac (and other members of its family), the prolyl oligopeptidases (POPs) from Basidiomycete mushrooms (AbPOPB and GmPOPB),¹⁶ and PCY1 from the plant Saponaria vaccaria (syn. Saponaria hispanica)¹⁷ are known to catalyse the formation of small macrocycles (containing between 5 and 9 amino acids). Although butelase 1 from Clitoria ternatea is extremely fast¹⁸ and highly versatile¹⁹ it does not produce small ring macrocycles efficiently²⁰, and a similar situation occurs with the MdnB from Microcystis aeruginosa (ATP-grasp superfamily)²¹. Further limiting the widespread use of butelase 1 at scale is the difficulty of its overexpression in E. coli¹⁸⁻¹⁹ and limited solubility (partially overcome by engineering but with decreased activity).²²

The prolyl oligopeptidase (POP) class of macrocyclases has been underexplored in terms of biocatalysis. The POPB from Basidiomycete fungi such as Amanita bisporigera and Galerina marginata (GmPOPB) species have been reported as having kcat values comparable to butelase, the fastest rate observed for peptide macrocyclisation. GmPOPB is the macrocyclase responsible for macrocyclisation of amatoxins, eight amino-acid ribosomal peptides with the core region IWGIGC(N/D)P. Amatoxins are cyclic peptides further modified by a characteristic sulfoxide cross-link between tryptophan and a cysteine, and hydroxylation (the extent of which vary). PCY1, the macrocyclase in the biosynthesis of a range of plant cyclic peptides known as segetalins, has been shown to be a naturally promiscuous enzyme capable to generate 5 to 9 residue segetalins.^{17, 26} PCY1 has been predicted to belong to the S9 protease family and homology modeling using a porcine muscle prolyl oligopeptidase (POP) structure (1QFS—protein bound to the inhibitor Z-proline-prolinal, 49% sequence identity) has been reported.^{17, 26}

### SUMMARY

The present inventors have undertaken detailed structural analysis of prolyl oligopeptidase (POP) macrocyclases and have unexpectedly found that they are able to efficiently generate macrocycles from precursor peptides that contain highly truncated C terminal recognition sequences. This may be useful for example in the biosynthetic production of macrocyclic molecules.

An aspect of the invention provides a method of producing a macrocyclic molecule comprising;
(i) providing a precursor molecule comprising a core region and a cyclisation tag of 10 or fewer amino acid residues; and,
(ii) reacting the precursor peptide with an isolated prolyl oligopeptidase (POP) macrocyclase to macrocyclise the core region and produce a macrocyclic molecule.

The precursor peptide may be reacted with the POP macrocyclase in the presence of a catalytic peptide. This may be useful for example in accelerating the rate of the macrocyclisation reaction.

Another aspect of the invention provides a precursor peptide comprising a core region and a cyclisation tag of 10 or fewer amino acid residues.

Another aspect of the invention provides a library of precursor molecules comprising a core region and a cyclisation tag of 10 or fewer amino acid residues, wherein the library comprises a diverse core region and the same cyclisation tag.

Another aspect of the invention provides a kit for use in producing a macrocyclic molecule comprising;
(i) a precursor peptide comprising a core region and a cyclisation tag of 10 or fewer amino acid residues, and
(ii) an isolated prolyl oligopeptidase (POP) macrocyclase.

A kit may further comprise a catalytic peptide, as described below.

Other aspects and embodiments of the invention are described in more detail below.

### BRIEF DESCRIPTION OF THE FIGURES

FIG. 1 shows the reaction scheme of macrocyclisation catalysed by PCY1 and sequence alignment of presegetalins. (a) amino acid one letter code is used to abbreviate amino acids, not depicted with the chemical formula. Transparent red circle is highlighting the peptide bond formed via macrocyclisation. (b) Red dots highlight end of core region. Sequence identity is highlighted with a gradient from white to blue. Deep blue showing highest sequence identity.

FIG. 2 shows a kinetic analysis of macrocyclisation catalysed by PCY1. (a) Comparative time course of PresegA1 (green), PresegB1 (blue) and PresegF1 (red) formation with PCY1. Data were fitted mono exponentially. b) LC/MS based steady state kinetics of PresegF1 with PCY1 using ion count quantification and product calibration curve, fitted non-exponentially. c) LC/MS based steady state kinetics of PresegB1 with PCY1 using quantification with UV at 280 nm, fitted non-exponentially. (d) Single injection ITC steady-state kinetics of PresegB1, fitted non-exponentially. Reaction temperature for all experiments: 30° C.

FIG. 3 shows binding studies of PCY1:S562A and presegetalins. (a) ITC curves showing equilibrium binding measurements for PresegA1 and PresegA1-NH2 with PCY1:S562A. (b) Thermodynamic parameters derived from equilibrium binding experiments for a selection of presegetalins. (c) Chemical structure of PresegA1 variants.

FIG. 4 illustrates the substrate scope of PCY1. Shorter substrates such as PresegF1-truncated and substrates containing non-amino acids are substrates for macrocyclisation catalysed by PCY1, yielding the cyclic peptides on the right.

FIG. 5 shows amanitin biosynthesis and the role of GmPOPB. (a) Biosynthesis of α-amanitin from the cyclic peptide produced by GmPOPB involves oxidation (blue circle), hydroxylations (purple circles), and cross linking (green circle). Amino acids in the cyclic peptide are depicted as geometrical shapes, and the core proline is labeled. (b) 35mer substrate used by GmPOPB to catalyse peptide bond hydrolysis and macrocyclisation. Peptide is divided in four regions, leader (blue), core (light grey), linker (magenta), and tail (orange). The colour coding for peptide regions for (c) follows this. (c) Scheme of the reactions catalysed by GmPOPB.

FIG. 6 shows the binding and kinetics of substrates and recognition tail truncations. (a) ITC binding curves for the 25mer peptide (obtained with the S577A mutant) and the 17 amino-acid recognition (linker and tail) sequence. (b) K_{d} values of peptides examined by ITC shows the 35mer and 25mer bind with similar affinity, the leader does not bind, the tail on its own binds weakly and the contributes to binding energy. The peptides are coloured as FIG. 4c with N-terminus at top. Error bars are standard error of the mean from the average of at least two independent measurements. (c) Michaelis-Menten curves show both the 13mer and 14mer are substrates for the enzyme. Error bars are standard error of the mean from duplicate measurements. (d)) Cyclic peptide produced after 1 h reaction with 1 μM GmPOPB and 200 μM of various peptide substrates. (e) Peptide produced after 16 h of reaction with 1 μM GmPOPB and 200 μM of various peptide substrates.

FIG. 7 shows steady state kinetics of PCY1 with presegetalin B1 and presegetalin F1. Native substrates presegetalin B1 and presegetalin F1 are rapidly processed by PCY1.

FIG. 8 shows steady state kinetics of PCY1 with the truncated presegetalin F1 substrates FSASYSSKPIQT and FSASYSSKPIQD in the presence and absence of catalyst peptide RNASAPV or DNASAPV. The presence of the catalytic peptide enhances the enzyme performance for short precursor peptides.

FIG. 9 shows a summary of the kinetics of the macrocyclisation of truncated presegetalin F1 substrates FSASYSSKPIQT and FSASYSSKPIQD in the presence and absence of catalyst peptide RNASAPV or DNASAPV. The presence of the catalytic peptide enhances the enzyme performance for short precursor peptides.

FIG. 10 shows a comparison of the efficiencies of PCY1 and PatGmac in the macrocyclisation of VTACITWP with different C-terminal cyclisation tags.

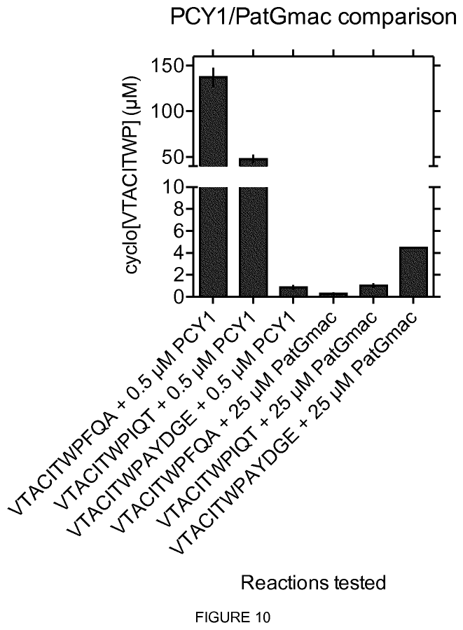

FIG. 11A shows the macrocyclisation of PresegF1 with various cyclisation tags using PCY1. FIG. 11B shows kinetics of PCY1 processing of peptide FSASYSSKPFQA. FIG. 11C shows kinetics of PCY1 processing of peptide FSASYSSKPIQT.

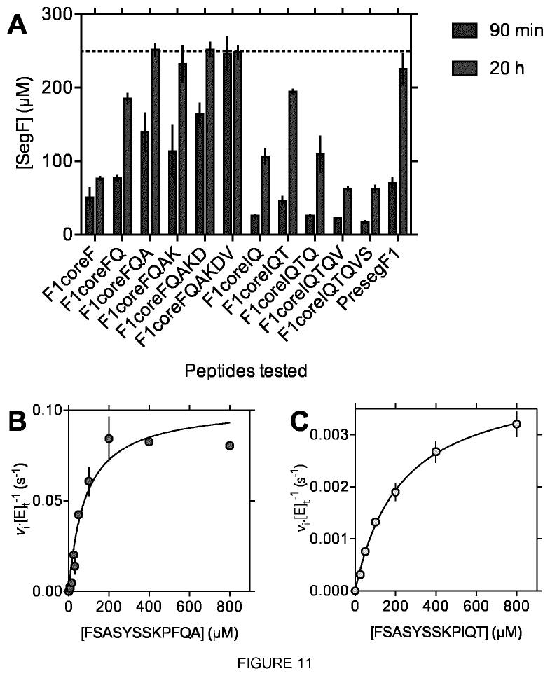

FIG. 12 shows examples of macrocyclic products molecules generated using PCY1.

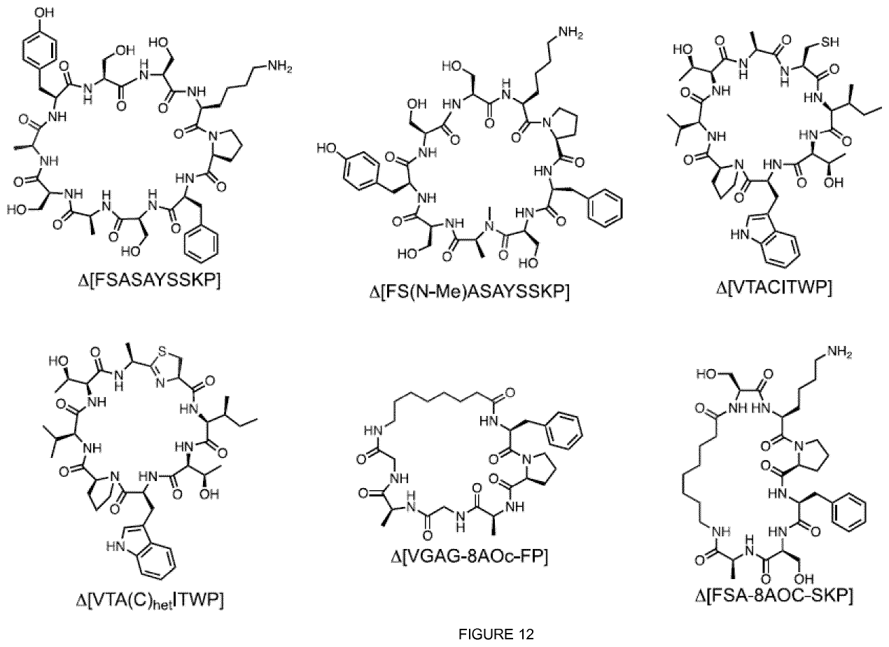

FIG. 13 shows a schematic representation of the interaction of the truncated presegetalin F1 substrate FSASYSSKPIQT and catalytic peptide RNASAPV (“split peptide”).

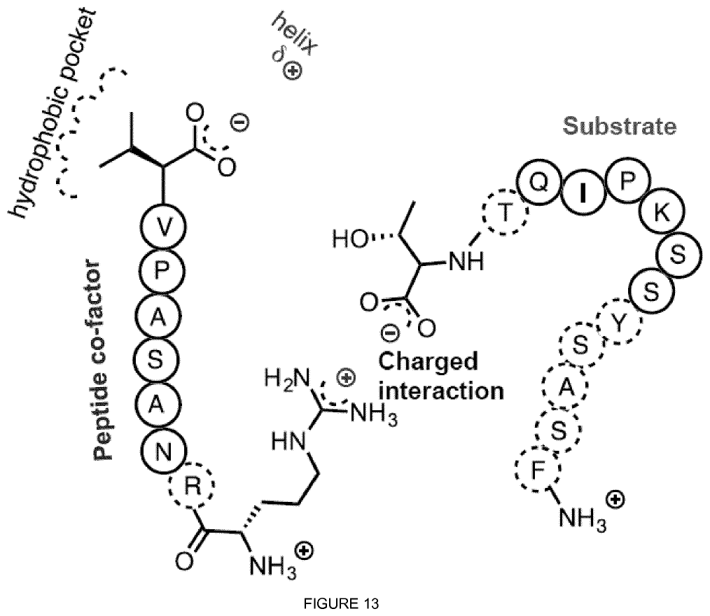

FIG. 14 shows a schematic representation of the interaction of the truncated presegetalin F1 substrate FSASYSSKPIQD and catalytic peptide RNASAPV (“split peptide”).

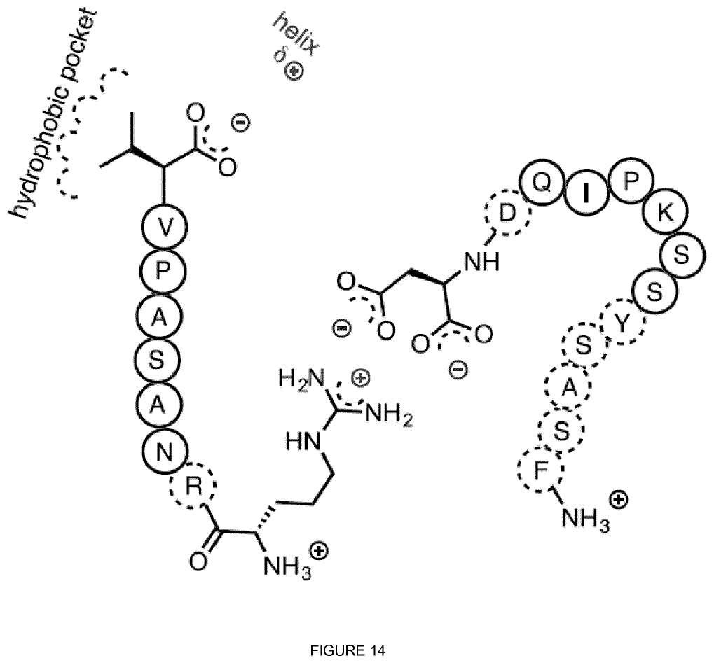

FIG. 15A shows the rates of reactions of PreF1coreT and PreF1coreD with catalytic peptides RNASAPV and DNASAPV. Catalytic peptide RNASAPV increases the rate of macrocyclisation of PreF1coreT. The wrong (mismatched) catalytic peptide (DNASAPV with PreFcoreD) reduces the rate of macrocyclisation. FIG. 15B shows the actual kinetics of reactions of PreF1coreT and PreF1coreD with catalytic peptides RNASAPV and DNASAPV.

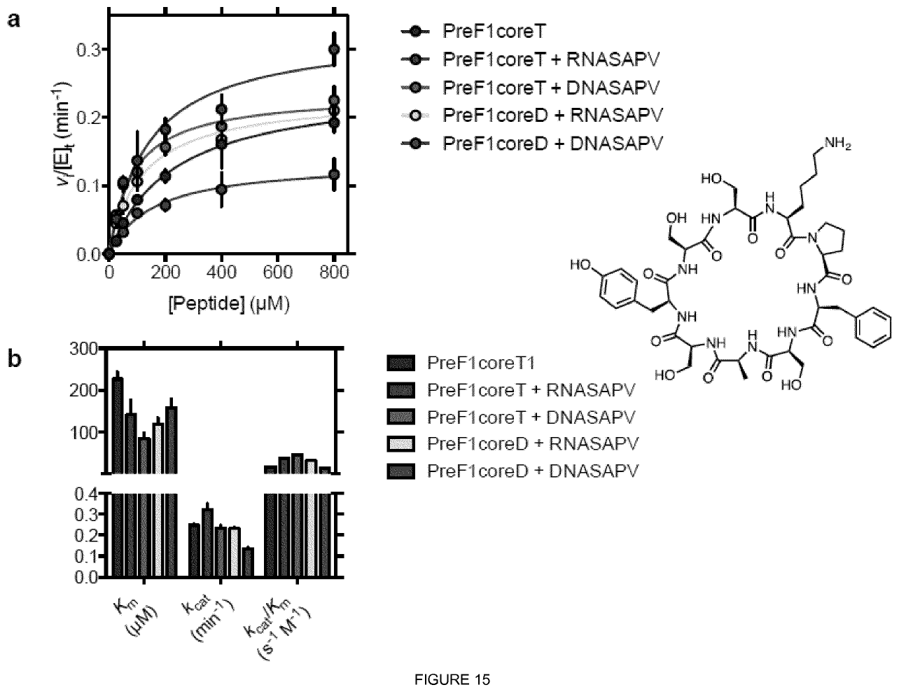

FIG. 16 shows the increase in the rate of PreF1coreT macrocyclisation in the presence of catalytic peptides RGNASAPV, RASAPV, RGGNASAPV, RANASAPV, RAANSAPV, RGANASAPV and RNASAPV.

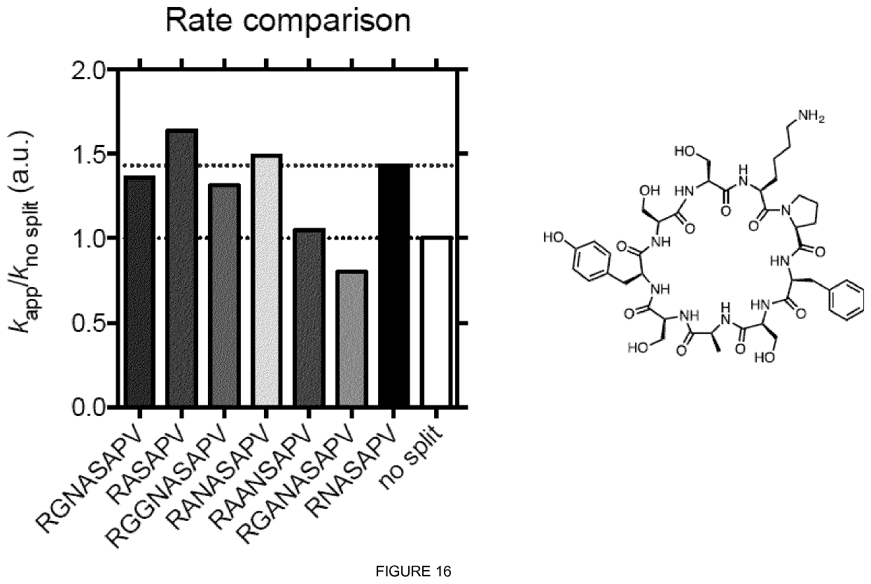

### DETAILED DESCRIPTION

This invention relates to the macrocyclisation of precursor peptides that comprise a cyclisation tag of 10 or fewer amino acid residues using a prolyl oligopeptidase (POP) macrocyclase. A short cyclisation tag as described herein may be useful in reducing the cost of synthesising precursor peptides and facilitating the generation of macrocyclic molecules by biosynthetic methods.

A prolyl oligopeptidase (POP) macrocyclase is an S9 serine peptidase of the POP family (PFA00326) that converts linear peptidyl substrates containing a C terminal recognition sequence into macrocycles. POP macrocyclases consist of two domains. The first domain consists of beta sheets and the second domain consists of a mixture of alpha helices and beta sheets. The C terminus of the linear peptidyl substrate may be anchored by the first (e.g. in GmPOPB) or the second (e.g. in PCY1) domain. POP macrocyclases may be identified by using standard sequence analysis software and/or using structural analysis tools, such as Protein Homology/analogY Recognition Engine (PHYRE: Kelley L A et al. Nature Protocols 10, 845-858 (2015), which can identify the characteristic arrangement of strands and helices that form the two domains of the POP macrocyclase. Active POP macrocyclases may also be identified by the presence of the of the serine protease catalytic triad of serine, histidine and aspartic acid residues.

Suitable POP macrocyclases include plant POP macrocyclases, such as PCY1 from Saponaria vaccaria (Barber et al J Biol Chem 2013, 288 (18), 12500-10), and fungal POP macrocyclases, for example Basidiomycete POP macrocyclases, such as AbPOPB from Amanita bisporigera and GmPOPB from Galerina marginata (Luo et al Chem Biol 2014, 21 (12), 1610-7). Other suitable POP macrocyclases are available in the art.

In some preferred embodiments, the POP macrocyclase may be a PCY1 macrocyclase. A PCY1 macrocyclase may have the amino acid sequence of Genbank database entry AGL51088.1 (UniProt accession number R4P353 (R4P353_9CARY); SEQ ID NO: 1) or a fragment or variant thereof. A PCY1 macrocyclase may be encoded by the nucleotide sequence of KC588970.1 or a fragment or variant thereof. Other PCY1 macrocyclase may have the amino acid sequence of SEQ ID NO: 2 of U.S. Pat. No. 9,394,561 and may be encoded by the nucleotide sequence of SEQ ID NO: 1 of U.S. Pat. No. 9,394,561. A variant of a PCY1 macrocyclase may comprise residues corresponding to D653, H695 and S562 of SEQ ID NO: 1.

In other embodiments, the POP macrocyclase may be a GmPOPB macrocyclase. A GmPOPB macrocyclase may have the amino acid sequence of Genbank database entry AEX26938.2 (SEQ ID NO: 2) or may be a fragment or variant thereof. A GmPOPB macrocyclase may be encoded by the nucleotide sequence of Genbank database entry JN827314.2 or a fragment or variant thereof. A variant of a GmPOPB macrocyclase may comprise residues corresponding to D661, H698 and S577 of SEQ ID NO: 2.

A variant of a reference amino acid sequence set out herein may have an amino acid sequence having at least 20%, at least 25%, at least 30%, at least 40%, at least 50%, at least 60%, at least 65%, at least 70%, at least 75%, at least 80%, at least 85%, at least 90%, at least 95%, at least 98% or at least 99% sequence identity to the reference amino acid sequence. Suitable reference amino acid sequences for POP macrocyclases, core regions and cyclisation tags are provided herein.

Amino acid sequence identity is generally defined with reference to the algorithm GAP (GCG Wisconsin Package™, Accelrys, San Diego Calif.). GAP uses the Needleman & Wunsch algorithm (J. Mol. Biol. (48): 444-453 (1970)) to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), generally employing default parameters.

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, up to 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:
(i) alanine and glycine;
(ii) glutamic acid, aspartic acid, glutamine, and asparagine
(iii) arginine and lysine;
(iv) asparagine, glutamine, glutamic acid and aspartic acid
(v) isoleucine, leucine and valine;
(vi) phenylalanine, tyrosine and tryptophan
(vii) serine, threonine, and cysteine.

A fragment is a truncated sequence which contains less than the full-length amino acid sequence but which retains some or all of the activity of the full-length amino acid sequence. For example, a fragment of a POP macrocyclase sequence may comprise at least 400 amino acids, at least 500 amino acids or at least 600 contiguous amino acids from the full-length POP macrocyclase sequence.

One or more heterologous amino acids, for example a heterologous peptide or heterologous polypeptide sequence, may be joined, linked or fused to a POP macrocyclase sequence set out herein

The POP macrocyclase reacts with a precursor peptide to generate a macrocyclic molecule. The precursor peptide is a linear molecule comprising or consisting of a core region and a cyclisation tag. The cyclisation tag of the precursor peptide is recognised by the POP macrocyclase, which then removes it from the precursor peptide and macrocyclises the core region to form a macrocyclic molecule.

The precursor peptide may comprise or consist of amino acid sequence (X₁ . . . Xₘ)(Y₁ . . . Yₙ), wherein X₁ to Xₘ are the core region and may be independently any chemical group; for example independently any amino acid; Y₁ to Yₙ are the cyclisation tag and may be independently any amino acid, m is 5-10 residues, preferably 5-9 residues, and n is 3-10 residues, preferably 3-6 residues.

The core region is the region of the precursor peptide which is macrocyclised by the POP macrocyclase to form the macrocyclic molecule. The core region is preferably located at the N terminus of the precursor peptide and is directly linked via a peptidyl bond at its C terminal end to the cyclisation tag.

The core region may be of any size that is suitable for macrocyclisation by the POP macrocyclase. A suitable core region may generate a macrocyclic molecule of 1500 Da or less. For example, the core region may consist of a chain of 30 or fewer members, 25 or fewer members, 20 or fewer members or 10 or fewer members. The core region may have 4 or more members, 5 or more members or 6 or more members.

The members of the core region chain may include amino acids. For example, a suitable core region may consist of a chain of 30 or fewer amino acids, 25 or fewer amino acids, 20 or fewer amino acids or 10 or fewer amino acids. The core region may have, 5 or more amino acids or 6 or more amino acids.

Suitable core regions for a PCY1 macrocyclase may consist of 5-10 members, preferably 8 members, for example 5-10 amino acids, preferably 8 amino acids. PCY1 macrocyclase is highly promiscuous and any core region of the appropriate size will be macrocyclised by PCY1. Preferably the core region macrocyclises to produce a macrocyclic molecule of 1500 Da or less. The core region of a precursor peptide for a PCY1 macrocyclase may be any natural or synthetic amino acid sequence.

Suitable core regions for a GmPOPB macrocyclase may consist of 5-10 residues, preferably 5 or 6 residues. Preferably the core region macrocyclises to produce a macrocyclic molecule of 1500 Da or less. In some embodiments, the core region for a GmPOPB macrocyclase may be an amatoxin sequence, such as IWGIGC(N/D)P or a variant thereof, for example a variant with 1, 2 or 3 conservative substitutions. Other examples of core regions are described below.

The core region of a precursor peptide may be a natural amino acid sequence, for example, a natural RIPP sequence or a precursor thereof; or the core region may be synthetic. The core region may include modified amino acids, unmodified amino acids, heterocyclic amino acids, non-heterocyclic amino acids, naturally occurring amino acids and/or non-naturally occurring amino acids. For example, the core region may comprise a non-naturally occurring amino acids selected from β-Ala, GABA, and Doc (8-amino-3,6-dioxaoctanoic acid. For example, the core region may comprise a heterocyclic amino acid selected from thiazoline (Thn), thiazole (Thz), oxazoline (Oxn), oxazole (Oxz), selenazolines, imidazolines, prolines and pseudoproline (ψPro). In some embodiments, heterocyclic amino acids may be introduced into the core region of the precursor peptide using isolated heterocyclase enzymes and optionally the oxidation of the introduced heterocyclic amino acids. A core peptide sequence may comprise 0, 1, 2, 3, 4, 5, 6, 7, 8 or more heterocyclic amino acids (Shin-ya, K. et al J. Am. Chem. Soc. 2001, 123, 1262-1263).

The members of the core region chain may include non-amino acid chemical groups. Suitable non-amino acid chemical groups include aryl rings, triazoles, such as 1,4-substituted 1,2,3-triazoles, polyethers, such as polyethylene glycol, alkyl groups, and polyketide chains, such as 7-aminoheptanoic acid (7Ahp) and 8-aminooctanoic acid (8Aoc). For example, a suitable core region may consist of a chain of 30 or fewer non-amino acid chemical groups, 25 or fewer non-amino acid chemical groups, 20 or fewer non-amino acid chemical groups or 10 or fewer non-amino acid chemical groups. The core region may have, 5 or more non-amino acid chemical groups or 6 or more non-amino acid chemical groups. Suitable non-amino acid chemical groups are described for example in Oueis et al, Angewandte Chemie International Edition 55 (19), 5842-5845; Oueis Chemistry Open 6 (1), 11-14; and Oueis et al ChemBioChem 16 (18), 2646-2650. A suitable core region may comprise one more non-peptidyl linkages, for example alkyl or other covalent linkages

The members of the core region chain may include both amino acids and non-amino acid chemical groups, as described above. For example, a core region may comprise 1, 2, 3, or more non-amino acid chemical groups.

In some embodiments, one or more residues in the core region may comprise a reactive functionality which may allow further chemical modification. Suitable residues may contain side chains with side chain linking groups such as NH2, COON, OH and SH.

The cyclisation tag is the region of the precursor peptide which is recognised by the POP macrocyclase. Upon recognition by the POP macrocyclase, the cyclisation tag is separated from the core region and the core region is macrocyclised to generate a linear peptide consisting of the cyclisation tag and a macrocyclic molecule. The cyclisation tag is located at the C terminus of the precursor peptide and is linked directly to the core region through a peptidyl bond.

In some less preferred embodiments, 1, 2, 3, 4 or 5 or more spacer residues may separate the core region and the cyclisation sequence in the precursor peptide.

C terminal recognition signals in natural substrates of POP macrocyclases generally consist in nature of 11 or more amino acids. The cyclisation tag described herein is a truncated POP macrocyclase recognition signal and may consist of 2-10 or 3-10 amino acid residues, preferably 3-6 residues, most preferably 3, 4, or 5 residues.

For reactions in the presence of a catalytic peptide, a cyclisation tag of 4-6 residues may be preferred.

In some embodiments, the cyclisation tag may be a fragment of a naturally occurring C terminal recognition signal, for example a fragment consisting of the 3-10 amino acid residues at the N terminal end of the recognition signal i.e. the 3-10 amino acids extending from the C terminus of the core region in a natural POP macrocyclase substrate.

In other embodiments, the cyclisation tag may be a variant of such a fragment, for example a fragment consisting of 1, 2 or 3 conservative substitutions, deletions or insertions relative to the sequence of the fragment. In some embodiments, the residue at the C terminus of the cyclisation tag may be mutated by substitution, deletion or insertion to optimise interaction with a catalytic peptide. For example, the C terminus residue may be replaced by a residue that has greater interaction with the N terminus of the catalytic peptide, such as increased salt bridge, hydrogen bond and/or van der Waal interactions.

In other embodiments, the cyclisation tag may be a synthetic sequence.

The sequence of the cyclisation tag may depend on the POP macrocyclase being used.

A suitable cyclisation tag for a PCY1 macrocyclase may consist of 2-6 or 3-6 residues. For example, a PCY1 cyclisation tag may consist of the N terminal 2-6 or 3-6 residues of the C terminal recognition signal of a presegetalin, such as presegA1, presegB1, presegD1 or presegF1, or a variant thereof. For example, the cyclisation tag may consist of IQTQVS, IQTQV, IQTQ, IQ(T/D) or IQ; AKDAEN, AKDAE, AKDA, or AKD; or FQAKDV, FQAKD, FQAK, FQA or FQ or a variant thereof. For example, a precursor peptide for reaction with a PCY1 macrocyclase may consist of the sequence (X₁ . . . Xₘ) IQT; (X₁ . . . Xₘ) IQD; (X₁ . . . Xₘ) AKD; or (X₁ . . . Xₘ) FQA, where X₁ to Xₘ are independently any chemical group, preferably any amino acid, and m is 5 to 10. Other suitable cyclisation tags for a PCY1 macrocyclase may consist of the N terminal 2-6 or 3-6 residues of the C terminal recognition signal of PatGmac, for example AYDGE, AYDG or AYD.

A suitable cyclisation tag for a GmPOPB macrocyclase may consist of 3-6 residues, preferably 5 or 6 residues. For example, a GmPOPB cyclisation tag may consist of the N terminal 5 or 6 residues of the C terminal recognition signal of the 35mer amatoxin peptide precursor, or a variant thereof. For example, the cyclisation tag may consist of WTAEH or WTAEHV or a variant thereof.

For example, the precursor peptide may comprise or consist of the sequence (X₁ . . . Xₘ)WTAEH or (X₁ . . . Xₘ)WTAEHV, wherein X₁ to Xₘ are independently any amino acid, Y₁ to Yₙ are independently any amino acid, and m is 5 to 9.

In some embodiments, the C terminal carboxyl group of the precursor peptide is replaced by an amide group.

One or more heterologous amino acids, for example a heterologous peptide or heterologous polypeptide sequence, may be joined or fused to a linear precursor peptide or other protein set out herein. For example a precursor peptide may comprise a precursor peptide as described above linked or fused to one or more heterologous amino acids.

In preferred embodiments, the POP macrocyclase may be reacted with the precursor peptide in the presence of a catalytic peptide.

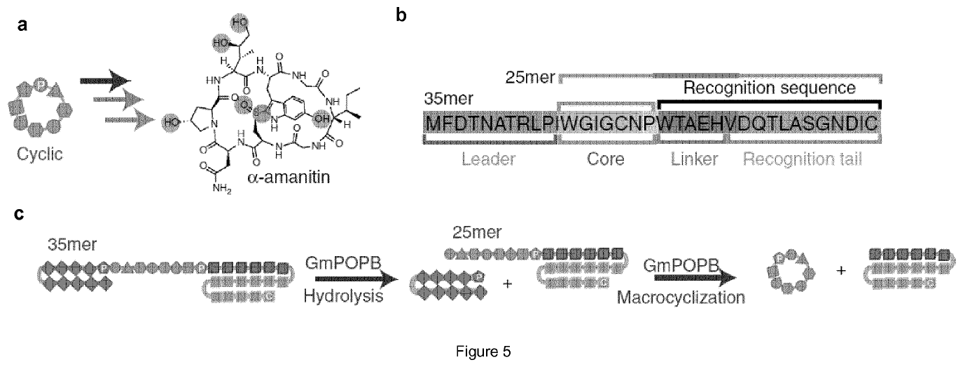

A catalytic peptide is a short linear peptide that consists of the C terminal 6-8 amino acids, preferably 7 amino acids, from the recognition tail of the wild-type linear POP macrocyclase precursor peptide or a variant thereof, for example with 1, 2 or 3 conservative substitutions. Variant amino acid sequences may be useful for example in optimising the solubility and/or binding of the catalytic peptide and reducing the cost of synthesis. The recognition tail of a POP macrocyclase precursor peptide may be readily identified by sequence analysis. For example, the recognition tail of the amanitin precursor is shown in orange in FIG. 5.

The catalytic peptide binds to the same binding site in the POP macrocyclase as the recognition tail of the wild-type linear POP macrocyclase precursor peptide and forms one or more non-covalent bonds through its N terminus with the C terminus of the precursor peptide.

A catalytic peptide may be generated using the POP macrocyclase structure and commonly available structural analysis software. In particular, the salt bridges, hydrogen bonds and/or van der Waal interactions that connect the precursor peptide and the catalytic peptide at the active site of the enzyme may be modeled to optimise the interaction. The sequence of the catalytic peptide, in particular its N terminus residue, may be optimised by structure determination in an iterative manner. For example, N-terminus of the catalytic peptide may have an unnatural amino acid or a non-amino acid to which enhances interaction with the precursor peptide. Similarly, in some embodiments, the substrate may have an unnatural amino acid or a non-amino acid at its C-terminus to enhance interaction with the catalytic peptide.

For example, an catalytic peptide for a PCY1 macrocyclase may be derived from the C terminus of the recognition tail of presegetalin F1, D1, A1 or B1 and may consist of the sequence (R/D/E)NAS(A/S)PV, for example RNASAPV or DNASAPV or may be a variant of any one of these sequences, for example a variant comprising 1, 2 or 3 conservative substitutions. In other embodiments, a catalytic peptide for a PCY1 macrocyclase may consist of the sequence (R/D/E)GNAS(A/S)PV, for example RGNASAPV; (R/D/E)AS(A/S)PV, for example RASAPV; (R/D/E)GGNAS(A/S)PV; for example RGGNASAPV; or (R/D/E)ANAS(A/S)PV, for example RANASAPV or may be a variant of any one of these sequences

An catalytic peptide fora GmPOPB macrocyclase may be derived from the C terminus of the recognition tail of the amanitin precursor and may consist of the sequence ASGNDIC or a variant thereof, for example a variant comprising 1, 2 or 3 conservative substitutions.

The precursor peptide and, optional catalytic peptide, may be produced by chemical synthesis or recombinant means as described below, and treated directly with the POP macrocyclase. This may be useful, for example in producing macrocyclic peptides which do not contain heterocycles or non-amino acid chemical groups.

The precursor peptide and catalytic peptide may be generated wholly or partly by chemical synthesis. For example, peptides and polypeptides may be synthesised using liquid or solid-phase synthesis methods; in solution; or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof. Chemical synthesis of peptides is well-known in the art (J. M. Stewart and J. D. Young, Solid Phase Peptide Synthesis, 2nd edition, Pierce Chemical Company, Rockford, Ill. (1984); M. Bodanzsky and A. Bodanzsky, The Practice of Peptide Synthesis, Springer Verlag, New York (1984); J. H. Jones, The Chemical Synthesis of Peptides. Oxford University Press, Oxford 1991; in Applied Biosystems 430A User's Manual, ABI Inc., Foster City, Calif.; G. A. Grant, (Ed.) Synthetic Peptides, A User's Guide. W. H. Freeman & Co., New York 1992, E. Atherton and R. C. Sheppard, Solid Phase Peptide Synthesis, A Practical Approach. IRL Press 1989 and in G. B. Fields, (Ed.) Solid-Phase Peptide Synthesis (Methods in Enzymology Vol. 289). Academic Press, New York and London 1997).

Non-natural residues and non-peptidyl linkages may introduced into the target molecule using standard chemical synthesis techniques.

Alternatively, a target molecule described herein may be generated wholly or partly by recombinant techniques. For example, a nucleic acid encoding the precursor peptide as described herein may be expressed in a host cell and the expressed precursor peptide isolated and/or purified from the cell culture.

Preferably, enzymes are expressed from nucleic acid which has been codon-optimised for expression in E. coli.

Nucleic acid sequences and constructs as described above may be comprised within an expression vector. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Suitable regulatory sequences to drive the expression of heterologous nucleic acid coding sequences in expression systems are well-known in the art and include constitutive promoters, for example viral promoters such as CMV or SV40, and inducible promoters, such as Tet-on controlled promoters. A vector may also comprise sequences, such as origins of replication and selectable markers, which allow for its selection and replication and expression in bacterial hosts such as E. coli and/or in eukaryotic cells.

Vectors may be plasmids, viral e.g. ‘phage, or phagemid, as appropriate. For further details see, for example, Molecular Cloning: a Laboratory Manual: 3rd edition, Russell et al., 2001, Cold Spring Harbor Laboratory Press. Many known techniques and protocols for expression of recombinant polypeptides in cell culture and their subsequent isolation and purification are known in the art (see for example Protocols in Molecular Biology, Second Edition, Ausubel et al. eds. John Wiley & Sons, 1992; Recombinant Gene Expression Protocols Ed R S Tuan (March 1997) Humana Press Inc).

In some embodiments, a POP macrocyclase, precursor peptide, catalytic peptide or other protein set out herein may be expressed as a fusion protein with a purification tag. Preferably the fusion protein comprises a protease recognition site between the enzyme sequence and purification tag. Following expression, the fusion protein may be isolated by affinity chromatography using an immobilised agent which binds to the purification tag. The purification tag is a heterologous amino acid sequence which forms one member of a specific binding pair. Polypeptides containing the purification tag may be detected, isolated and/or purified through the binding of the other member of the specific binding pair to the polypeptide. In some preferred embodiments, the tag sequence may form an epitope which is bound by an antibody molecule.

Various suitable purification tags are known in the art, including, for example, MRGS(H)₆, DYKDDDDK (FLAG™), T7-, S-(KETAAAKFERQHMDS), poly-Arg (R₅₋₆), poly-His (H₂₋₁₀, poly-Cys (C₄) poly-Phe(F₁₁) poly-Asp(D₅₋₁₆), Strept-tag II (WSHPQFEK), c-myc (EQKLISEEDL), Influenza-HA tag (Murray, P. J. et al (1995) Anal Biochem 229, 170-9), Glu-Glu-Phe tag (Stammers, D. K. et al (1991) FEBS Lett 283, 298-302), Small Ubiquitin-like Modifier (SUMO) tag or a Hiss-SUMO tag (Marblestone et al Protein Sci. 2006 January; 15(1): 182-189), Cherry tag (Eurogentec), Tag.100 (Qiagen; 12 aa tag derived from mammalian MAP kinase 2), Cruz tag 09™ (MKAEFRRQESDR, Santa Cruz Biotechnology Inc.), glutathione-S-transferase and Cruz tag 22™ (MRDALDRLDRLA, Santa Cruz Biotechnology Inc.). Known tag sequences are reviewed in Terpe (2003) Appl. Microbiol. Biotechnol. 60 523-533. The TAG sequence may be linked to the target protein through a protease recognition site, for example a TEV protease site, to facilitate removal following purification. After isolation, the fusion protein may then be proteolytically cleaved to produce the precursor peptide, or other protein set out herein.

The precursor peptide may be treated with the POP macrocyclase under suitable conditions for the macrocyclisation of the core region. Suitable conditions may be readily determined by those skilled in the art. Examples of suitable conditions may include 500 mM NaCl and/or pH 9. For example, the linear precursor peptide substrate may be treated with the POP macrocyclase in 500 mM NaCl and 5% DMSO at pH 8.

The highest temperature tolerated by the macrocyclase is generally preferred as this leads to increased reaction rates. The optimal temperature for reaction under a defined set of conditions may be determined experimentally.

In some embodiments, the precursor peptide or the prolyl oligopeptidase (POP) macrocyclase immobilised on a solid support. For example, the precursor peptide may be immobilised, for example on a solid support, and the POP macrocyclase may be free in solution. This may be useful, for example in facilitating purification of the macrocyclic peptide. Alternatively, the precursor peptide may be free in solution and the POP macrocyclase may be immobilised for example on a solid support, such as a bead. This may be useful, for example in facilitating re-cycling of the POP macrocyclase.

Macrocyclic molecules produced by the methods described herein may include macrocyclic peptides and derivatives thereof. The macrocycle may comprise amino acids, non-amino acid chemical groups or a mixture of amino acids and non-amino acid chemical groups. The linkages in the macrocycle may be peptidyl linkages, non-peptidyl linkages or a mixture of peptidyl linkages and non-peptidyl linkages. Suitable macrocyclic molecules include natural and synthetic macrocycles of 1500 Da or less.

Preferred macrocyclic molecules include macrocyclic peptides. Macrocyclic peptides may be N-to-C linked peptides and may be synthetic or naturally occurring molecules, such as ribosomally synthesized and post-translationally modified peptides (RiPPs), including macrocycles containing thiazoline or oxazoline or combinations thereof. Examples of naturally occurring macrocyclic peptides include amatoxins, phallotoxins, cyclotides and cyanobactins, for example patellamides, ulithiacyclamides, trunkamides, and telomestatins. Patellamides are cyclic octapeptides produced by Prochloron spp which include patellamide A, B, C and D. Examples of synthetic macrocyclic molecules are described in Oueis et al, Angewandte Chemie International Edition 55 (19), 5842-5845; Oueis et al Chemistry Open 2015 6 (1), 11-14; and Oueis et al ChemBioChem 16 (18), 2646-2650

The precursor peptide may be subjected to chemical modification before macrocyclisation and/or macrocyclic molecule may be subjected to further chemical modification after macrocyclisation. Suitable modifications include for example oxidation, hydroxylation, epimerisation, cross-linking and/or prenylation. Methods of modifying macrocyclic molecules are well known in the art.

Suitable modifications also include derivatisation with a heterologous moiety, for example, a moiety containing a natural side group such as OH, NH2, COON, SH, or an unnatural side group suitable for coupling reactions and click chemistry. The use of orthogonal reactive groups, such as azidoalanine A(N₃) or a dehydroalanine (Dha), to introduce chemical diversity is reported in Oueis et al (2015) supra.

Click-chemistry typically involves the Cu(I)-catalysed coupling between two components, one containing an azido group and the other a terminal acetylene group, to form a triazole ring. Since azido and alkyne groups are inert to the conditions of other coupling procedures and other functional groups found in peptides are inert to click chemistry conditions, click-chemistry allows the controlled attachment of almost any linker to the macrocyclic peptide under mild conditions. For example, non-cyclised cysteine residues of the macrocyclic peptide may be reacted with a bifunctional reagent containing a thiol-specific reactive group at one end (e.g. iodoacetamide, maleimide or phenylthiosulfonate) and an azide or acetylene at the other end. Label groups may be attached to the terminal azide or acetylene using click-chemistry. For example, a second linker with either an acetylene or azide group on one end of a linker and a chelate (for metal isotopes) or leaving group (for halogen labeling) on the other end (Baskin, J. (2007) PNAS 104(43)16793-97) may be employed.

The macrocyclic molecule may be labeled with a detectable label.

The detectable label may be any molecule, atom, ion or group which is detectable in vivo by a molecular imaging modality or other means. Suitable detectable labels may include metals, radioactive isotopes and radio-opaque agents (e.g. gallium, technetium, indium, strontium, iodine, barium, bromine and phosphorus-containing compounds), radiolucent agents, contrast agents and fluorescent dyes.

The choice of detectable label depends on the molecular imaging modality which is to be employed. Molecular imaging modalities which may be employed include radiography, fluoroscopy, fluorescence imaging, high resolution ultrasound imaging, bioluminescence imaging, Magnetic Resonance Imaging (MRI), and nuclear imaging, for example scintigraphic techniques such as Positron Emission Tomography (PET) and Single Photon Emission Computerised Tomography (SPECT).

The macrocyclic molecule may be attached to an antibody molecule, such as an antibody or antibody fragment or derivative, for example for use in antibody-directed drug therapies. Suitable techniques for the conjugation of macrocyclic molecules and antibodies are well known in the art.

Following production, the macrocyclic molecule may be isolated and/or purified. Suitable methods for purifying macrocyclic molecules are well known in the art.

The methods of the invention are suitable for the production of usable amounts of macrocyclic molecules, such as macrocyclic peptides. Following production as described above, the macrocyclic molecule may be isolated and/or purified and used as required. Alternatively, the macrocyclic molecule may be used without further isolation or purification. Macrocyclic molecules produced as described herein, such as macrocyclic peptides or other biomolecules, may be useful in therapeutics, nanotechnology applications and in optical/electronic or contractile materials.

An isolated element exists in a physical milieu distinct from that in which it occurs in nature, or in which it was produced recombinantly. For example, an isolated peptide may be substantially isolated with respect to the complex cellular milieu in which it naturally occurs. The absolute level of purity is not critical, and those skilled in the art can readily determine appropriate levels of purity according to the use to which the protein is to be put.

A heterologous element is an element which is not associated or linked to the subject feature in its natural environment i.e. association with a heterologous element is artificial and the element is only associated or linked to the subject feature through human intervention.

In some embodiments, the POP macrocyclase and/or precursor peptide may be immobilised on a solid support.

A solid support is an insoluble, non-gelatinous body which presents a surface on which the peptides or proteins can be immobilised. Examples of suitable supports include glass slides, microwells, membranes, or beads. The support may be in particulate or solid form, including for example a plate, a test tube, bead, a ball, filter, fabric, polymer or a membrane. A peptide or protein may, for example, be fixed to an inert polymer, a 96-well plate, other device, apparatus or material. The immobilisation of peptides and proteins to the surface of solid supports is well-known in the art.

In some embodiments, the amount of other species of molecule in the reaction products (i.e. linear species that have not been macrocyclised) may be undetectable, for example by HPLC analysis. In other embodiments, the reaction products may comprise residual amounts of other species, such as precursor peptide or linear core peptides.

In some embodiments, the homogenous product may be purified and/or isolated after macrocyclisation. In other embodiments, no purification or isolation of the reaction product may be required after macrocyclisation.

Macrocyclic molecules produced as described herein may be screened for biological or other activity.

For example, a method of screening a macrocyclic peptide library may comprise;
(i) providing a population of precursor peptides, each precursor consisting of a core region and a cyclisation tag having 10 or fewer amino acids, wherein the core region is diverse in the population and the cyclisation tag is the same,
(ii) treating said population with a prolyl oligopeptidase (POP) macrocyclase to convert the core region into a macrocyclic molecule,
(iii) screening the macrocyclic molecules for activity, and
(iv) identifying an active macrocyclic molecule.

In some embodiments, a precursor peptide may be immobilised on a bead. A reference copy of said precursor peptide may be additionally immobilised to said bead, said reference copy lacking a cyclisation signal. The bead may be treated with a POP macrocyclase as described herein, such that the precursor peptide is macrocyclised and the generated macrocyclic molecule released from the bead, while the reference precursor peptide lacking the cyclisation tag remains immobilised to the bead. The macrocyclic molecule may be screened to identify a biological activity. The bead which released the macrocyclic may then be identified and the reference copy immobilised on said bead sequenced to identify the macrocyclic molecule with the biological activity. For example, a method of screening a macrocyclic molecule library may comprise;
(i) providing a diverse population of core regions attached to beads, each bead having a first and a second copy of the core regions attached thereto, wherein the first copy but not the second copy is attached to the bead via a cyclisation tag,
(ii) treating said beads with a prolyl oligopeptidase (POP) macrocyclase to convert the first copy of the core region into a macrocyclic molecule and release the macrocyclic molecules from the beads,
(iii) screening the macrocyclic molecules for activity,
(iv) identifying an active macrocyclic molecule
(v) identifying the bead from which the macrocyclic molecule was released, and
(vi) sequencing the second copy of the core region attached to the bead.

The population of core peptides may be spatially arrayed, such that the bead from which the macrocyclic peptide was released can be identified.

Other aspects of the invention provide materials, reagents and kits and reagents for use in the production of macrocyclic peptides and populations thereof and the use of such macrocyclic peptides, for example in screening methods. These materials may include a precursor peptide consisting of a core region and a cyclisation tag as described above and a library of precursor peptides consisting of a core region and a cyclisation tag as described above, wherein the core region comprises diverse residues at 1, 2, 3 or more positions.

The precursor peptides in the library may be immobilised on magnetic beads.

In some embodiments, a split and pool approach may be used to generate a macrocyclic molecule library for screening. Suitable split and pool approaches for the generation of diverse peptide sequences on beads are well known in the art.

For example, a pool of beads containing an immobilised cyclisation tag may be split into portions and a different first amino acid is coupled to the N terminal of the cyclisation tag in each portion. The portions are pooled and re-split into portions and a different second amino acid is coupled to the first amino acid in each portion. These steps are repeated until a full length precursor peptide is immobilised to each bead.

Along with the precursor peptide, a reference copy of the core region of that precursor protein may also be immobilised on to each bead. This allows the identification of a core region of interest by sequencing the reference copy as described above. The reference copy may be generated during the split and pool synthesis by coupling amino acids directly to the bead as well as the immobilised cyclisation tag.

Other aspects of the invention provide a vector comprising a nucleic acid sequence encoding a precursor peptide as described above; and a vector comprising a cloning site for insertion of a core region and a nucleic acid sequence encoding a cyclisation tag as described above downstream of the cloning site, such that the vector expresses a precursor peptide comprising a core region inserted into the cloning site and the cyclisation tag.

Another aspect of the invention provides a population of vectors that expresses a library of precursor peptides as described above.

Another aspect of the invention provides a kit comprising a precursor peptide or library of precursor peptides as described above or nucleic acid encoding said precursor peptide or library.

A kit may further comprise an isolated POP macrocyclase, for example a PCY1 or GmPOPB macrocyclase. Suitable POP macrocyclases are described above.

A kit may further comprise a catalytic peptide, as described above.

A kit may further comprise buffers, additional enzymes, solubilising agents, stabilising agents, oxidants, antioxidants, and enzyme co-factors, such as ATP and FAD.

The POP macrocyclase and/or precursor peptide may be immobilised on a solid support, such as a magnetic bead or multi-well plate.

A kit may further comprise a multi-well plate;
each individual well containing a homogenous population of core regions attached to beads,
each bead having a first and a second copy of the core region attached thereto, wherein the first copy but not the second copy is attached to the bead via a cyclisation signal,
the sequences of the core regions being different in different wells.

The kit may include instructions for use in a method of producing a macrocyclic molecule as described above.

A kit may include one or more other reagents required for the method, such as buffer solutions, solid supports, and purification reagents.

A kit may include one or more articles for performance of the method, such as means for providing the test sample itself, including sample handling containers (such components generally being sterile).

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Experimental

1. Materials and Methods

1.1 Cloning, Expression and Protein Purification

Codon optimized full-length PCY1 (Saponaria vaccaria) including an N-terminal His6-tag and a cleavable Tabacco etch virus (TEV) protease site (sequence ENLYFQ/G) encoded in pJ414 plasmid was purchased from DNA2.0. Overexpression of PCY1 was performed in E. coli BL21 (DE3) grown in LB media. Cultures were inoculated and grown at 37° C. until OD600 nm: ˜0.6, after which the temperature was lowered to 16° C. and protein expression was induced with 0.5 mM IPTG (final concentration, Generon). Bacteria were grown for 20 h at 16° C., and cells were harvested by centrifugation at 8,983 g for 10 min at 4° C. (Avanti J-26S, rotor JLA-8.1000, New Brunswick Scientific). Wet cell pellets were stored at −80° C. until protein purification. Cell pellets were resuspended in buffer A (50 mM HEPES, 300 mM NaCl, 10% (v/v) glycerol, 3 mM bME (Fischer Chemical), pH 8.0) supplemented with complete EDTA-free protease inhibitor tablets (Roche) and 0.4 mg DNase I from bovine pancreas per g wet cells, at 4° C. Cells were disrupted by double passage through a cell disruptor (Constant Systems Ltd) at 30 kPSI. The lysate was subsequently cleared via centrifugation at 43,667 g at 4° C. for 20 min (rotor JA 25.50, New Brunswick Scientific). The cleared lysate was loaded onto a 5 mL Co HiTrap TALON (GE Healthcare), pre-equilibrated in buffer A. Protein was eluted in buffer B (50 mM HEPES, 300 mM NaCl, 10% (v/v) glycerol, 250 mM imidazole, 3 mM bME, pH 8.0). TEV protease was added to the eluted sample (1 mg per 30 mg of eluted protein) and this mixture was dialyzed into 1 L of buffer A at 4° C. overnight. The dialyzed sample was applied onto a 5 mL Co HiTrap TALON, equilibrated in buffer C (50 mM HEPES, 300 mM NaCl, 10% (v/v) glycerol, 3 mM bME, pH 8.0). The flow-through of this reverse Ni-affinity chromatography step was pooled and dialyzed into 1 L of buffer D (50 mM HEPES, 5 mM NaCl, 10 (v/v) glycerol, 3 mM bME, pH 8.0) at 4° C. for 1 h and subsequently subjected to anion exchange chromatography using a 5 mL HiTrap Q-FF column (GE Healthcare) and a gradient of buffer D and E (50 mM HEPES, 500 mM NaCl, 10% (v/v) glycerol, 3 mM bME, pH 8.0). Eluted samples containing PCY1 (verified by SDS-PAGE) were pooled, concentrated and further purified using size-exclusion chromatography (HiLoad 16/600 Superdex 200 pg, GE Healthcare) pre-equilibrated in buffer F (50 mM HEPES, 150 mM NaCl, 10% (v/v) glycerol, 1 mM TCEP (tris(2-carboxyethyl)phosphate), pH 8.0). Purity and identity of final protein sample were confirmed by SDS-PAGE and mass spectrometry.

The plasmid pJExpress414 encoding the codon optimized G. marginata POPB gene was purchased from DNA 2.0. Plasmids were transformed into BL21(DE3) cells (Agilent). Cultures (50 mL) were grown overnight at 37° C. in the presence of 100 μg/mL ampicillin, then diluted 100-fold into 6 L Terrific Broth (TB) media. These cultures were grown at 37° C. with shaking (200 rpm) until the optical density at 600 nm (OD600) reached 0.6. Cells were cooled down for 1 h to 16° C., and protein expression was then induced by the addition of 0.5 mM isopropyl β-D-thiogalactopyranoside (IPTG, Generon). Cultures were incubated for an additional 16 h and centrifuged at 6000×g for 15 min. Cell pellets were resuspended in 250 mL Ni-NTA lysis/wash buffer A (50 mM HEPES pH 8.0, 300 mM NaCl, 10 mM imidazole, 10% glycerol, and 2 mM β-mercaptoethanol) supplemented with complete EDTA-free protease inhibitor tablets (Roche Applied Science). The resulting suspension was lysed by two passages through a cell homogenizer at 30,000 psi, and purified by nickel chromatography. Each desired protein was eluted using a step elution with lysis buffer supplemented with 250 mM imidazole (buffer B). Eluted protein was dialyzed overnight against buffer C (50 mM HEPES (pH 8.0), 50 mM NaCl, 10% glycerol, and 2 mM β-mercaptoethanol) while simultaneously the His-tag was cleaved by TEV protease. This dialyzed TEV-cleaved mixture was loaded onto a Histrap column connected in tandem to a Hitrap Q-FF column. Both columns were washed with buffer C, and GmPOPB was eluted during this wash. Fractions were pooled and concentrated to <8 mL (at 10 mg/mL approximately). Protein was loaded onto a Superdex S200 gel filtration column (GE Healthcare) pre-equilibrated with storage buffer D (50 mM HEPES (pH 8.0), 50 mM NaCl, 10% glycerol, and 2 mM β-mercaptoethanol). Fractions containing pure protein were combined, concentrated, divided in aliquots, flash frozen, and stored at −80° C. Protein concentrations were determined by absorbance at 280 nm.

1.2 Site Directed Mutagenesis

Site directed mutagenesis was performed with the method of Liu et al.³² using an overlaying mutation carrying primer pair (IDT) of ˜30 nucleotides used for PCR with Pfu polymerase (Thermo Scientific), according to manufacturer instructions. The PCR product was DpnI digested (Thermo Scientific, according to manufacturer's instructions), and used for transformation of E. coli DH5a. Sequencing was performed by Eurofins.

1.3 General Procedure for Kinetic Assays.

Comparison between distinct GmPOPB substrates was performed in 50 mM Tris pH 8.0, 50 mM NaCl, 10 mM DTT with varying concentrations of substrates at room temperature. All reactions were performed in duplicates. Reactions were started by adding GmPOPB (50 nM for GmAMA1_C6S, 1 μM for the 13mer and 14mer, and 20 nM for other substrates) to the assay mixture containing buffer and peptide. Reactions were quenched at several time points by adding 50 μL reaction mixture to 20 μL 6% TFA. Reactants were separated from products for quantification by injecting 50 μL of each quenched time point mix onto a ZORBAX SB-C18, 5 μm, 9.4×50 mm (Agilent) column connected to an Agilent LC-MS (G6130B Single Quad, Agilent Technologies). Reactants were separated from products using a gradient from H2O containing 0.1% TFA or 0.1% formic acid and 5% acetonitrile to 50% acetonitrile, at 1.5 ml/min for 8 min. Peaks with ultra violet (UV) absorbance at 220 and 280 nm were integrated, the area of peaks corresponding to reactant and products was used to calculate the percentage of product formed after a correction for differences in the extinction coefficient of each peptide was applied (ε280-25mer=11,000M-1 cm-1, ε220-25mer=46,000M-1 cm-1, ε280-cyclic=5500M-1 cm-1, ε220-cyclic=34,000M-1 cm-1, and ε280-tail=5500M-1 cm-1). The sum of product+substrate was assumed equal to the total initial amount of substrate, product converted from % to concentration. This value was divided by concentration of enzyme present to yield v/Et (min-1). When enzyme mutants and peptides containing alanine in the core region were tested for activity, higher concentrations 5 μM enzyme and 100 μM substrate were incubated for 1 and 18 h at room temperature. For progress curves with the 35mer substrate, measurements were triplicate and quantification relied on ion counts from mass spectrometry. Mass signals corresponding to 35mer (1282.9 Da-M+3H), 25mer (900.7 Da-M+2H), leader peptide (1165.5 Da-M+H), recognition sequence (930.4 Da-M+2H), macrocyclic peptide (841.3 Da-M+H), linear peptide (859.4 Da-M+H) were monitored, the area of each was integrated and quantified using a calibration curve performed with the 25mer, 35mer, cyclic, and linear peptides as standards. Authentic macrocyclic peptide was quantified by UV absorbance. Data showing products formed after 1 and 16 h with truncated peptides were performed twice. UV and ion count approaches gave similar results for the 25mer. Kinetic data were fitted to a Michaelis-Menten equation using GraphPad Prism, and values reported are average and standard error of the mean.

1.4 Crystallization, Data Collection and Crystallographic Analysis

PCY1 and PCY1:S562A crystals (apo and as complex) were obtained from hanging drop vapor diffusion crystallization experiments with 500 μL reservoir solution and 2 μL drops (1:1 protein/precipitant ratio). When additive screens were performed, 0.2 μL of additive screen (Hampton Research) was added to the 2 μL crystallization drop. Crystals were harvested, cryo protected and subsequently flash frozen in liquid nitrogen. All data sets were collected at Diamond Light Source (UK). Apo PCY1 was crystallized at 12.2 mg/mL in 33% (w/v) PEG 2000, 113.75 mM Mg formate and 0.1 M Na cacodylate pH 7.0. For cryo protection 10% (v/v) glycerol were added to reservoir solution. Data was collected at 100 K at beamline i04. Data was processed using the processing pipeline xia2 3d33. PCY1:S562A complex with PresegA1 was crystallized at 13.3 mg/mL with 163 μM PresegA1 in 28% (w/v) PEG 5000 MME, 140 mM Mg acetate tetrahydrate and 0.1 M Tris pH 7.5. For cryoprotection 7% (v/v) glycerol were added to reservoir solution. Data was collected at 100 K at beamline i04-1. Data was processed using the processing pipeline xia2 3dii.

PCY1:S562A complex with PresegB1 was crystallized at 13.3 mg/mL with 163 μM PresegB1 in 34.5% (w/v) PEG 3350 and 70 mM Mg sulfate. For cryo-protection 5% (v/v) glycerol were added to reservoir solution. Data was collected at 100 K at beamline i04-1. Data was processed using the processing pipeline xia2 3d33. PCY1:S562A complex with PresegF1 was crystallized at 13.0 mg/mL with 161.3 μM PresegB1 in 27% (w/v) PEG 3350, 100 mM Calcium chloride and 0.1 M Bis-Tris at pH 6.5. For cryo-protection 10% (v/v) glycerol were added to reservoir solution. Data was collected at 100 K at beamline i24. Data processing was performed with xia233 for apo/A1/B1/F1. Apo PCY1 structure was solved using molecular replacement using PHASER and 1QFS as search model (α/β-hydrolase and β-propeller domain as separated search models). The initial solution was then completed with buccaneer and manual model building and refinement of the model were performed using Coot and refmac, respectively, including TLS refinement38 and model validation using MolProbity. For all co-complex crystal structures, the apo PCY1 structure was used as search model.

ApoGmPOPB crystals were obtained by vapor diffusion at 20° C. using the hanging drop method. The initial conditions in the drop were 100 mg/mL GmPOPB, 30% PEG4000, and 100 mM MES buffer, pH 6.5. Several crystal clusters appeared after incubation at 20° C. for 1 week, which were crushed and used for microseeding using a 80 mg/mL GmPOPB solution and the same precipitant. Crystals were cryoprotected by addition of 10% glycerol to precipitant solution, and flash cooled in liquid nitrogen. All complex structures were obtained by vapor diffusion at 20° C. using the sitting drop method. Complexes with both 25mer and 35mer peptides were obtained by co-crystallization of 100 mg/ml protein and two-fold molar excess of peptide, and contained 12.5 mM Hexamine cobalt chloride as additive. For the S577A-25mer complex, crystals were obtained with 28% PEG6000, 100 mM Bicine pH 9.0, 60 mM magnesium formate, and 2.42% DMSO. Crystals were cryoprotected by addition of 12% glycerol to precipitant solution, and flash cooled in liquid nitrogen. For the D661A-25mer complex, crystals were obtained with 28% PEG6000, 100 mM Tris pH 8.3, and 90 mM sodium/potassium phosphate. Crystals were cryoprotected by the addition of 12% glycerol to precipitant solution, and flash cooled in liquid nitrogen. Crystals of S577A-35mer complex were obtained with 28% PEG6000, 100 mM Bicine pH 8.7, 64 mM sodium potassium phosphate. Crystals were cryoprotected by addition of 12% glycerol to precipitant solution, and flash cooled in liquid nitrogen. For the H698A-25mer complex, crystals were obtained with 27% PEGMME2000, 90 mM Bicine pH 8.7, and 100 mM potassium thiocyanate. Crystals were cryoprotected by addition of 13% glycerol to precipitant solution, and flash cooled in liquid nitrogen. Data were collected at 100 K at the European Synchrotron Radiation Facility (ESRF) beamline ID30A-3 (5577A-25mer complex), Diamond Light Source beamlines I02 (apo GmPOPB), 104-1 (5577A-35mer complex), I03 (D661A-25mer), or in house on a Rigaku 007HFM rotating anode X-ray generator with a Saturn 944 CCD detector (H698A-35mer). Data were processed with HKL2000 (5577A-25mer and H698A-35mer complexes) or Xia2-DIALS (apo GmPOPB, 5577A-35mer and D661A-25mer complexes). All structures were solved by molecular replacement with PHASER, followed by density improvement using PARROT, then automatic building using Buccaneer and Arp/wARP. Manual rebuilding was performed with COOT, and refinement was performed with REFMAC5 implemented in the CCP4 program suite, Phenix, and PDB_REDO. Structural figures were generated with PyMOL (DeLano Scientific, LLC). The solution structures for 13mer, 25mer, and 35mer free were generated by PEPFOLD and the macrocyclic peptide was adapted from a-amanitin (PDB: 3CQZ).

1.5 ITC Studies with PCY1 Variants and Peptide Ligands

All titrations were performed on a MicroCal PEAQ-ITC instrument (MicroCal, Malvern Instruments, Northampton, Mass., USA) and the results were fitted with PEAQ-ITC analysis software (MicroCal, Malvern Instruments, Northampton, Mass., USA).

All PCY1 samples were buffer-exchanged or solubilized into buffer F and further diluted in the same batch of buffer F, in order to minimize buffer mismatches. ITC binding experiments were performed using a cell solution containing PCY1:S562A and a titrant solution with each peptide used. All peptides were used at 200 μM except for presegetalin A1-NH2 (700 μM). The cell contained 20 μM, 18.3 μM, 17.1 μM 15.5 μM and 100 μM PCY1:S562A for Presegetalin A1, B1, D1, F1 and A1-NH2, respectively. The measurements were performed at a stir speed of 750 rpm at 25° C. using a reference power of 10 μcal/s, except for A1-NH2 (5 μcal/s). A buffer titration control in which peptide was titrated into buffer was performed for each experiment. Data processing, fitting to a non-linear one-site binding model, and data plotting was performed using MicroCal PEAQ ITC analysis software (Malvern). Single injection kinetic ITC measurements were performed using MicroCal PEAQITC with 60 nM PCY1 in the syringe and 193 μM presegetalin B1 in the cell. To start the reaction, 2 μL of 9.06 μM PCY1 was injected into the cell (4 s injection time) containing 300 μL of 193.5 μM Presegetalin B1. The measurement was performed in buffer at 30° C., at 750 rpm using a reference power of 5 μcal/s.

GmPOPB peptide ligand solutions were prepared in 20 mM Tris pH 8.0 containing 1 mM DTT, prior to buffer exchange by three cycles of dilution in 50 mM Tris pH 8.0 with 50 mM NaCl, 10 mM DTT followed by concentration using a Microsep Advance centrifugal device equipped with a 1 kDa cut off membrane (Pall Corporation). The same three cycles of dilution in 50 mM Tris pH 8.0 with 50 mM NaCl and 10 mM DTT followed by concentration were performed with the protein to be used in the titration using a Vivaspin protein concentrator spin column with a 30 kDa cut off (GE Healthcare). A final dilution to the concentration to be used for titration was performed using the buffer that passed through during the protein buffer exchange, both for the protein and peptide to be used to avoid any possible buffer mismatch. The stirred cell contained 300 μL of protein (the inactive mutant GmPOPB_S577A at 20 μM for 35mer, 36 μM for 10mer, 36 μM for 11mer, 29 μM for 12mer, 42 μM for 13mer, 29 μM for 14mer, 37 μM for 9mer recognition sequence, 21 μM for 12mer recognition sequence), and the injection syringe contained 75 μL of peptide ligand (200 μM for 35mer, 924 μM for 10mer, 761 μM for 11mer, 484 μM for 12mer, 442 μM for 13mer, 582 μM for 14mer, 1 mM for 9mer recognition sequence, 677 μM for 12mer recognition sequence). Titrations of peptide into protein solutions were conducted at 20° C. For all the titration experiments, a total of 19 injections of 2 μL were made at 120 s intervals. The heat released due to the first injection (0.4 μL) was excluded from data analyses. Binding data with the H698A mutant were performed by titrating enzyme (319 μM stock) into 25mer peptide (27 μM). Blank runs in which peptide (or H698A) was titrated into buffer were performed to correct for the heats of dilution and mixing, and the dilution isotherm for each peptide ligand was subtracted from the respective binding isotherm prior to curve fitting. Equilibrium dissociation constants (K_{d}) as well as ΔH and ΔS values for binding of each peptide to protein were obtained by fitting the calorimetric data with a single-site model using the stoichiometry parameter n fixed at 1.0 using Malvern PEAQ-ITC data analysis software.

We performed all ITC binding experiments at least in duplicate, and calculations of average and standard error of the mean were performed with GraphPad Prism.

1.6 LC/MS Assay: Time Courses and Steady-State Kinetics

All kinetic measurements (activity tests, progress curves and initial-rate experiments) were performed as discontinuous assays analyzing samples via LC/MS or MALDI (10-mer activity test). Reactions were performed in 20 mM Tris pH 8.5, 100 mM NaCl, 5 mM DTT (for progress curves 0.2 mg mL-1 BSA was included).17 Reactions were performed at 30° C. and at various time points 50 μL samples were quenched with 20 μL of 6% (v/v) TFA. Samples (50 μL) were loaded onto a ZORBAX SBC18, 5 μm, 9.4 mm×50 mm (Agilent) column connected to an Agilent LC-MS instrument (G6130B Single Quad, Agilent Technologies). Reactants were separated from products using a gradient from A (H2O with 0.1% formic acid) to 80% B (acetonitrile), at a rate of 1.5 mL/min for 8.75 min. For data analysis, ion count peaks and UV absorbance peaks were integrated using Agilent ChemStation software. For data analysis involving quantitative mass spec integration, calibration curves with macrocyclic peptide product were performed in triplicate in order to linearly correlate ion counts to cyclic product concentration. Data fitting and plotting was performed with Prism 6.

1.7 Comparison of Catalytic Peptide Steady State Kinetics

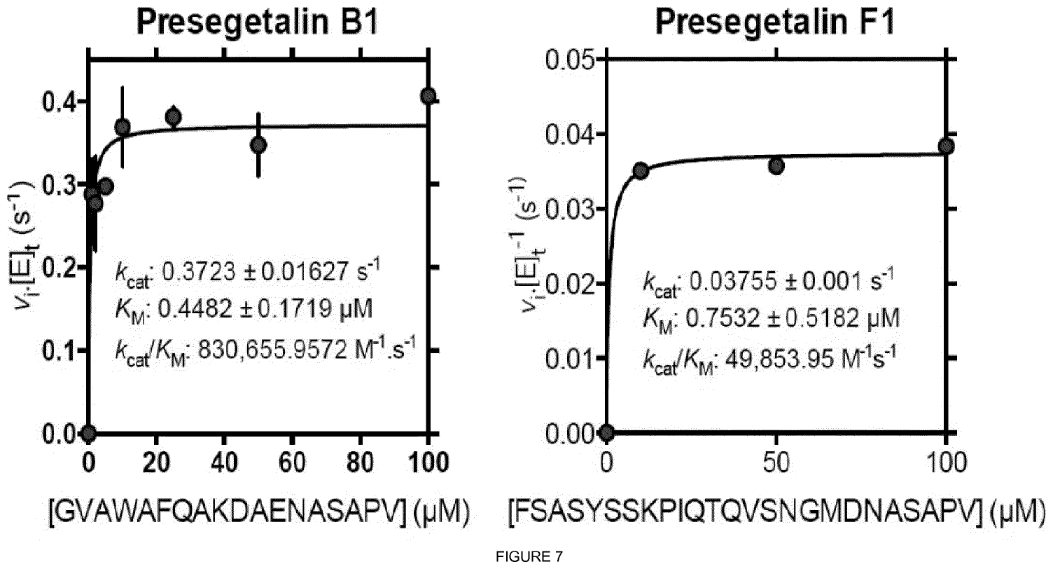

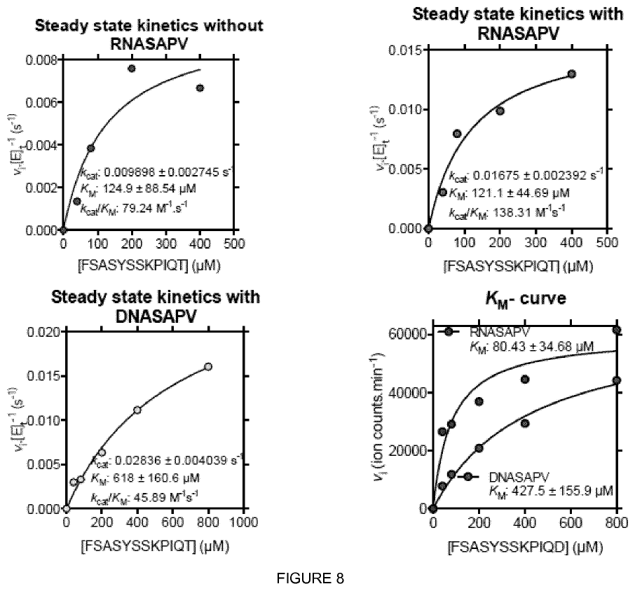

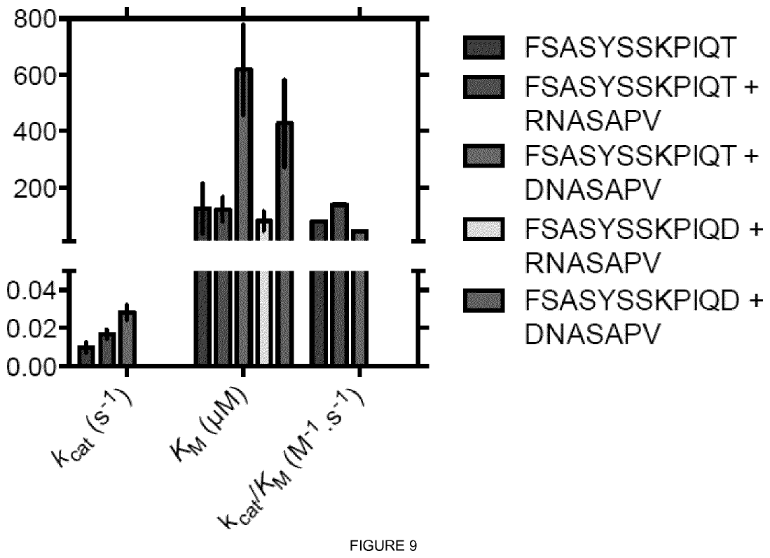

Initial rate measurements at different substrate concentrations were performed in order to derive steady state kinetics. All reactions were performed with 0.25 μM PCY1 and 25, 50, 100, 200, 400 or 800 μM substrate (FSASYSSKPIQT or FSASYSSKPIQD). Reactions containing catalytic peptide RNASAPV or DNASAPV were supplemented with 500 μM of each peptide, respectively. Reactions were performed in 20 mM TRIS pH 8.5, 100 mM NaCl, 5 mM DTT at 30° C. Time points were taken at 5, 15, 30, 45 and 60 min. Samples (50 μL) at each time point were quenched with 20 μL of 6% (v/v) TFA and then spun down for 30 min at 3,080×g. Samples were analysed via LC/MS (ZORBAX SBC18, 5 μm, 9.4 mm×50 mm (Agilent) column connected to an Agilent LC-MS instrument (G6130B Single Quad, Agilent Technologies)) using 0.1% (v/v) formic acid and a gradient of acetonitrile (5%-95%). Extracted ion count peaks for corresponding product (A[FSASYSSKP]) masses ([M]: 954.44, [M]+H⁺: 955.44 and [M]+Na⁺: 977.44) were integrated and converted in corresponding product concentration using an ion count v. [product] calibration curve. Product concentration time courses were analysed with linear regression and slopes were plotted as a function of substrate concentrations (Michaelis Menten plot), deriving steady state kinetic parameters shown in FIGS. 7 to 9.

1.8 Synthesis of Peptides Containing Non-Amino Acids

The HPLC grade acetonitrile (MeCN) was purchased from Fisher. Aqueous buffers and aqueous mobile-phases for HPLC were prepared using water purified with an Elga® Purelab® Milli-Q water purification system (purified to 18.2 MΩ.cm) and filtered over 0.45 μm filters. Solvents, amino acids and coupling reagents were purchased commercially from different sources and used without any further purification. Automated solid-phase peptide synthesis (SPPS) was carried out on a Biotage® Syro Wave™ system in polypropylene (PP) syringe with a PTFE frit. Final cleavage and deprotection were completed manually. The different precursor peptides were synthesized by standard automated solid-phase (SPPS) on a Chem-Matrix Rink amide resin (˜0.5 mmol/g) using the Fmoc strategy and Fmoc-protected amino acids. A double coupling strategy using a 5-fold excess with HBTU/DIEA (HBTU, 0.5M in DMF and DIEA, 2M in NMP) and DIC/oxyma pure (DIC, 0.5M in DMF, Oxyma, 1M in DMF) was used for all amino acids for 30 minutes at 75° C. The Fmoc deprotection was done in 20% piperidine/DMF for 12 min at rt. For the final cleavage and side chain deprotection, the beads (washed with CH2Cl2 and dried) were transferred into a flacon tube and the cleavage cocktail was added and left shaking for 2 h: 96% TFA, 2.5% H2O, 1.5% TIS. The resin was filtered, washed with CH2Cl2, and the filtrate concentrated under reduced pressure. The peptides were then precipitated in cold Et2O and the precipitate purified by prep-HPLC. Semi-preparative RP-HPLC was performed on an Agilent Infinity 1260 series equipped with an MWD detector using a Macherey-Nagel Nucleodur C18 column (10 μm×21×250 mm at 21 mL/min) and fractions were collected automatically by peak detection at the specified wavelength using an Agilent 1260 Infinity preparative-scale fraction collector using the following chromatographic system: MeCN and 0.1% aqueous TFA [95% TFA (5 min), linear gradient from 5 to 95% of MeCN (35 min), 95% MeCN (40 min)] and UV detection at 280 nm for peak collection, while monitoring at the additional two wavelengths 220 and 254 nm. Fractions of the pure peptides were combined and freeze dried before being used for the assays. Analytical RP-HPLC-MS was performed on an Agilent infinity 1260 series equipped with a MWD detector using a Macherey-Nagel Nucleodur C18 column (10 μm×4.6×250 mm) and connected to an Agilent 6130 single quad apparatus equipped with an electrospray ionization source using the following chromatographic system.: 1 mL/min flow rate with MeCN and 0.1% aqueous TFA [95% TFA (5 min), linear gradient from 5 to 95% of MeCN (35 min), 95% MeCN (40 min)] and UV detection at 220 nm.

FSA-8Aoc-SKPIQT-NH2, yield=70%; MS (ESI+) m/z (%): [M+2H+] 560.0 (100), [M+H+] 1118.6 (50), [M+Na+] 1140.4 (5); HPLC tR=12.80 (purity=95%). VGAG-8Aoc-FPIQT-NH2, yield=62%; (ESI+) m/z (%): [M+H+] 1029.6 (100), [M+2H+] 515.4 (75), [M+Na+] 1051.5 (5); HPLC tR=17.00 (purity=99%). 8Aoc (8-aminooctanoic acid).

2. Results

2.1 PCY1

2.1.1 Mechanism of PCY1

PCY1 operates via an acyl enzyme intermediate in a classical serine protease mechanism²⁶ with the N-terminus of the peptide rather than water acting as the nucleophile. The PatGmac macrocyclase also operates by this mechanism,²⁷ although possessing an entirely different fold and being part of a distinct enzyme family. The putative catalytic triad (D653, H695 and S562) is located in the peptide binding site of PCY1. The catalytic serine and histidine are further apart than is observed for the triad in other POPs, although a similar ‘non-optimal’ arrangement is present in GmPOPB. Activity tests using PresegB1 as a substrate for macrocyclisation showed that S562A and H695A were inactive.

2.1.2 Kinetic Characterization of PCY1

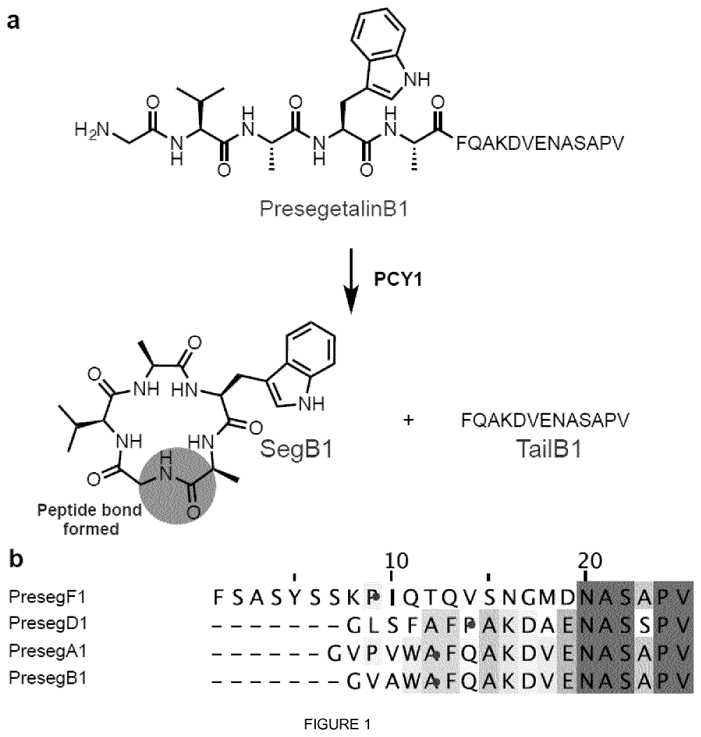

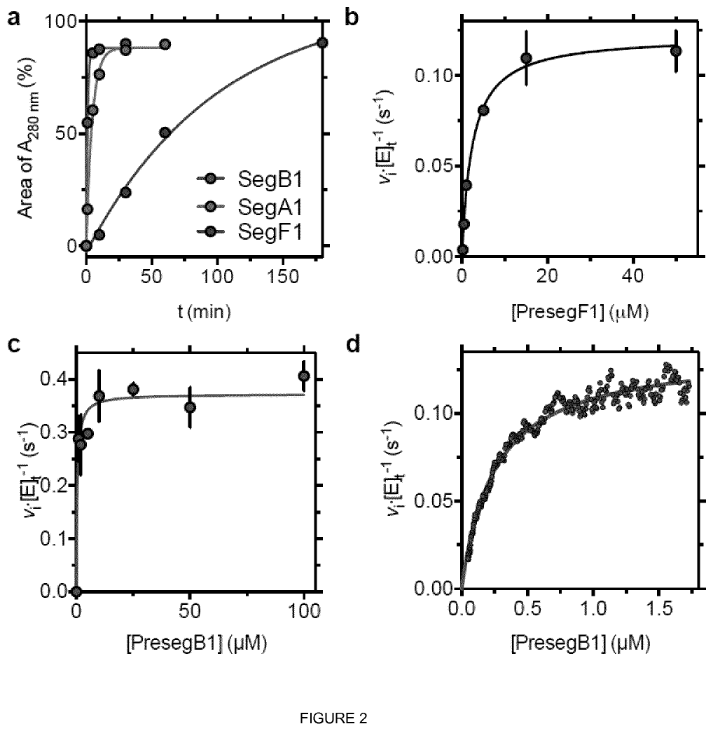

PCY1 extracted from S. vaccaria seeds has been previously described to have a turnover number of ˜1 h⁻¹ for PresegA1.¹⁷ The enzyme produced heterologously in E. coli was evaluated by LC/MS-based time course experiments macrocyclizing 200 μM of PresegA1, PresegB1 or PresegF1 with 3.6 μM PCY1. These substrates were chosen as they produce 6, 5 and 9 residue macrocycles, respectively, show variability in the length of the C-terminal tail sequence, and have a different C-terminal core residue (proline in PresegF1, alanine in the other two) (FIG. 1b). PresegB1 was completely macrocyclized in ˜30 min, PresegA1 in ˜50 min and PresegF1 in >3 h (FIG. 2a). We chose to investigate the slowest and fastest substrate by steady-state kinetics using an LC/MS assay and UV280 nm quantification. This resulted in a k_{cat}/Kₘ value of 830,000 M-1 s⁻¹ for PresegB1 (FIG. 2c and Table 1), which is not only considered above ‘average’ for a typical enzyme28 but is also larger than the efficiency using the second-order rate constant of butelase 1 from Clitoria ternatea (542,000 M-1 s⁻¹), the fastest macrocyclase described to date.¹⁸ The UV280 nm detection at low substrate concentrations and thus accurate determination of Kₘ was problematic, especially for PresegF1, which lacks a tryptophan in its sequence. We explored steady-state kinetics with single injection kinetic ITC for PresegB1 (FIG. 2d and Table 1) and this allowed more accurate determination of Kₘ (0.25±0.01 μM). Under the ITC conditions, the measured k_{cat} (0.14±0.01 s⁻¹) was a factor of three lower than the LC/MS derived values. However, to obtain kinetic parameters for PresegF1, a different approach was required since single injection kinetic ITC only showed a small heat change. Therefore, we resorted to an LC/MS assay followed by quantification using mass ion counts, relying on a calibration curve employing the macrocyclic peptide product as a standard (FIG. 2b). This approach gave a Kₘ of 2.40±0.51 μM and k_{cat} of 0.12±0.01 s⁻¹ for PresegF1. These data show that the difference in efficiency that we observed in the time course experiments is due to large differences in Kₘ rather than in the turnover rate.

2.1.3 Crystallization of PCY1

We determined the structure of PCY1 to a resolution of 2.55 Å employing molecular replacement using porcine POP model (pdb: 1QFS). The structure of apo PCY1 displays the overall architecture of the POP family comprising an α/β-hydrolase domain and a seven bladed β-propeller. A cacodylate molecule from the crystallization buffer was found in the cavity located at the interface of the a/bhydrolase domain and the β-propeller domain, adjacent to the presumed active site comprising S562, H696 and D563. Cacodylate is coordinated through hydrogen bonds by the side chain hydroxyl group of Y481 and the amide of N563; both residues have been proposed to form the oxyanion hole in the POP-family of enzymes.²⁹

2.1.4 Substrate Recognition

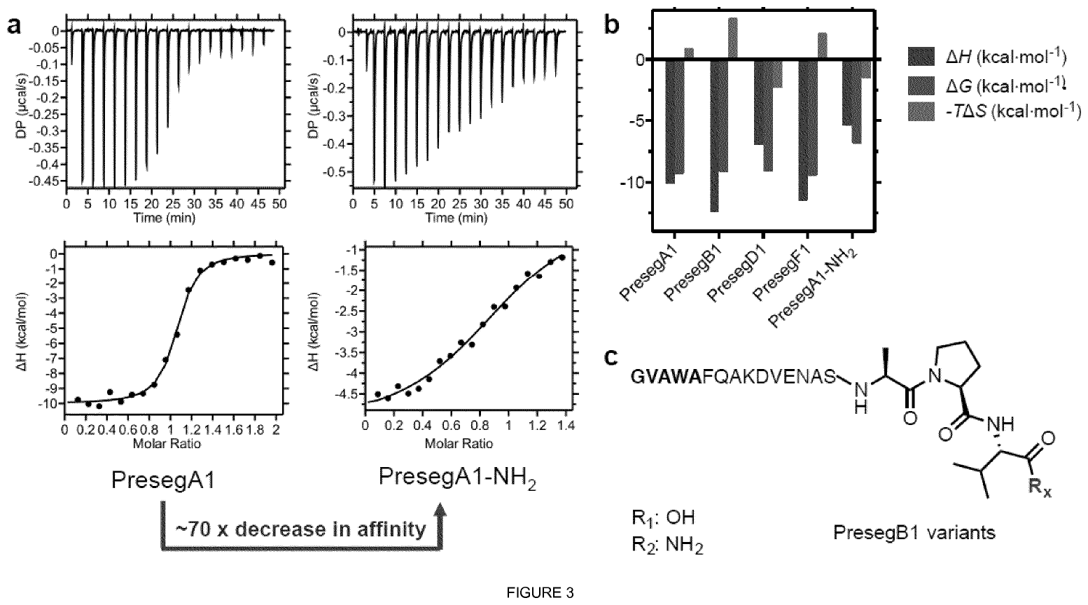

We determined complex crystal structures of PCY1:S562A bound to PresegA1 (2.0 Å), PresegB1 (2.17 Å) and PresegF1 (1.86 Å). We did not observe continuous electron density spanning the entire length of the peptide substrate in any of the structures solved. In all three complexes however, the C-terminal peptide (NASA(S)PV) was clearly visible and adopts the same structure. In the PresegF1 complex, the highest resolution structure, the carboxylic acid group of the substrate's C-terminus V25 makes hydrogen bonds to the backbone amide and side chain of S495 as well as the side chain of S493. The carboxyl group is positioned at the N-terminal end of the helix thus interacting with the helical dipole. In the substrate, the side chains of Val25, Pro24, Ala23, and Ala21 make only a few van der Waal contacts with the protein, while the main chain of Ala21 makes hydrogen bonds with the protein. Both the main and side chain of Ser22 and Asn20 make either direct or water bridged hydrogen bonds. In the PresegA1 complex, no other residues could be modeled. However, in PresegF1 and PresegB1 complex structures, it was possible to model the core peptide in at least one of the subunits within the asymmetric unit, although the residues that connect it to the tail were absent. In PresegF1, we visualized Phe1 to Gln11, but not Thr12 to Met19. In the other three subunits, less of the core peptide could be modeled suggesting there could be multiple binding conformations. In the best-ordered subunit, one face of the proline (Pro9) ring in the substrate stacks against W603 whilst the edge of the ring interacts with F484. The Leu8-Pro9 peptide bond (P2-P1) adopts a trans configuration. The remainder of the core peptide makes a small number of hydrogen bonds and van der Waal interactions. The scissile bond P1-P1¢ (Pro9-Ile10) is positioned such that the modeling of the hydroxyl at S562A results in the distance and orientation expected for the formation of the acyl enzyme intermediate, with the Y481 acting to stabilize the negative charge. The positioning of the scissile bond at the active site validates the biochemical relevance of the structure. The P1¢ side chain (Ile10) sits in a hydrophobic cleft making contacts with the side chains of Y481 and I466. The amino terminus of the substrate is over 20 Å from the scissile bond, requiring extensive conformational flexibility from the substrate for macrocyclisation to occur. The only other POP acting as macrocyclase whose structure has been determined is GmPOPB (described below).²³ A comparison between complex structures of PCY1-S562A bound to PresegF1 and GmPOPB-S577A bound to a 35 amino acid substrate (GmAMA1) reveal that although the overall protein architecture is remarkably similar, significant differences exist between the location of the peptide ligand, and positioning of specific regions of each peptide. In GmPOPB, the entire peptide tail or recognition sequence (which is 17 amino acids long) can be seen in the structure, and spans through the β-propeller domain. In contrast, in PCY1 the recognition sequence is not inserted into the propeller domain, and only the last C-terminal residues can be observed, demonstrating greater flexibility in the peptide region that follows the core peptide, which is not the case in GmPOPB. Additionally, in GmPOPB the core region is compressed in the center of the protein, while in PCY1 the core region occupies a more extended position akin to the one occupied by the leader peptide in GmPOPB. The core region of the macrocyclisation substrate was not observed in GmPOPB (pdb codes 5N4B and 5N4D)²³. In the PresegB1 complex, the P2-P1-P1¢ residues (Trp4, Ala5 and Phe6) adopt an identical main chain conformation, make similar interactions as PresegF1, and are thus positioned for attack by S562. However, the remaining three residues of the core peptide have a different and more ring-like conformation than PresegF1 and as a result the N-terminus is only 11 Å away from the scissile bond. Thus, PresegB1 needs much less re-organization than PresegF1 and this may underlie the difference in their macrocyclisation turnover rates. Comparing the PCY1 structures in the PresegB1 and PresegF1 complexes reveals the structures are very similar (root mean square deviation 0.8 Å over 707 Ca atoms). The most notable change occurs in the main chain at Y696 which, in the PresegF1 complex, has flipped so that the side chain points into the structure core, and as consequence the loop at E154 has moved to accommodate it. In PresegB1, the residue adopts a different rotamer but the main chain is unchanged. The change at Y696 appears necessary to avoid a clash between this residue and the core of the substrate. In both complexes, R655 makes hydrogen bonds to the substrate in the P2-P1-P1¢ region although the precise interactions are different. Informed by the crystal structures, we constructed mutants S493A, S495A, Y696G, R655A and W603A using the reaction with PresgB1 as a test substrate. The relative activities of S495A, S493A, R696G, and R655A were decreased by half, while W603A and H695Q showed around 10% relative activity, and H695A was inactive. No variant resulted in an increase in proteolytic (as opposed to macrocyclase) activity. To further investigate substrate binding, we performed ITC experiments using PresegA1, PresegB1, PresegD1 and PresegF1 with PCY1:S562A. All four peptides showed tight binding with K_{d} values around 200 nM (FIG. 3a). The major contribution to binding of all tested peptides is enthalpic rather than entropic (FIG. 3b), pointing towards interactions between protein and substrate rather than by the exclusion of water. The C-terminal tail of PresegA1 was previously reported to be essential for activity.¹⁷ The only conserved amino acids in all natural substrates are the C-terminal six residues (NASA(S)PV). The peptide NASAPV binds with a K_{d} of only 25 μM suggesting that this region although conserved does not dominate binding. We measured the binding of PresegA1-NH2 (an amide rather than carboxylic acid at C-terminus) to PCY1:S562A and observed a ˜70-fold decrease of affinity (K_{d}: 12.1 μM) (FIG. 3). Our data point to a key role for W603 in positioning the substrate with the correct geometry for attack by S562. Unlike the PatGmac class of macrocyclasemacrocyclases,²⁷ PCY1 does not require a proline residue or cis-like geometry for binding and cleavage. Instead, the site of macroyclization is controlled by the nature of the residues at P1 and P1¢ of the substrate. The structure shows that PCY1 residues W603, F484, Y607, and V558 clash with the amino acids that have two non-hydrogen substituents at Cb (Val, Ile, Thr) or larger. Although serine cannot be ruled out on purely steric grounds, the hydrophobic nature of the environment favors proline or alanine. The binding site at P1¢ is hydrophobic but would require larger side chains such Ile, Phe, or Leu to make interactions with the protein. This sequence pattern is indeed found at the point of scission for all known substrates for PCY1. Although our data show and rationalize the importance of the carboxy C-terminus, the significance of its conservation pattern is less clear. Indeed, the binding energy of just the six residues of the tail peptide is relatively low compared to the substrate. A substrate peptide without the C-terminus (PresegF1-truncated) was evaluated for binding and its K_{d} was too high to be accurately measured due to poor peptide solubility at high concentrations. The structures show that the two most ordered regions of the substrate are the P2-P1-P1¢ of the core and the C-terminus. The peptide that links these regions is highly variable in sequence and in length (FIG. 1b) consistent with this region acting as a flexible linker rather than a recognition element. Whilst the intact peptide binds very tightly, these two regions on their own bind weakly, suggesting a model where affinity is generated by linking weakly binding sites (the extremely well known chelate effect).³⁰

2.1.5 Exploring PCY1 Substrate Promiscuity

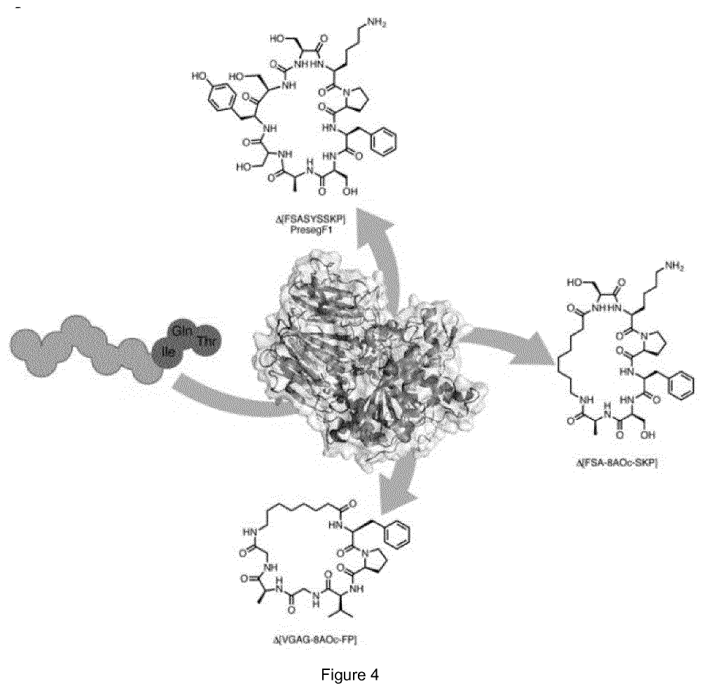

It is known that PCY1 is tolerant of significant changes in sequence within the core peptide and competent to make macrocycles as short as five and as large as nine residues.¹⁷ We tested the ability of PCY1 to make larger macrocycles by extending the core region of PresegF1 by one amino acid and PCY1 was able to macrocyclize this substrate. This extends the full range of relevant peptide macrocycles that PCY1 makes to include the upper limit for ‘beyond rule of five’ macrocycles, with typical molecular weight around 1000 Da.³¹ The requirement for a long disposable C-terminus is a major drawback of the approach. We therefore investigated the ability of PCY1 to process a peptide with a short C-terminal extension. PCY1 was able to process a peptide with a very short C-terminal extension (PresegF1-truncated, extension IQT, FIG. 4) with synthetically useful k_{cat} and Kₘ values when compared to PatGmac (Table 1, Kₘ=227 μM, k_{cat}=0.052 s⁻¹ for PCY1, while the PatGmac has Kₘ values around 50 μM and kcat between 1 h⁻¹ and 1 day⁻¹ depending on the substrate used). Although GmPOPB23 can also process short tailed substrates, it is slower (k_{cat} at 20° C. using a shorter substrate is 0.010 s⁻¹). We also investigated whether PCY1 was capable of macrocyclizing substrates containing non-amino acids; a desirable property for a useful chemical tool. After 16 h incubation with PCY1 of the hybrid precursor peptides FSA-8Aoc-SKPIQT-NH2 and VGAG-8Aoc-FPIQT-NH2 containing a seven carbon alkyl chain, the corresponding hybrid macrocyclic peptides shown in FIG. 4, were produced. The first peptide is a derived analogue of PresegF1, while the other is an analogue derived of a PatGmac substrate.¹³ The Maldi spectrum shows about a 50% conversion rate overnight for the first peptide FSA-8AOc-SKPIQT with only traces of the linear-cleavage peptide alongside the macrocyclic peptide, while the other precursor peptide VGAG-8Aoc-FPIQT is almost entirely converted overnight to a mixture of the corresponding macrocyclic peptide and linear-cleavage peptide. The same reaction with the highly promiscuous PatGmac would have required 60 times the amount of enzyme and an average of 20 days for completion. Nonetheless, PatGmac usually gives rise to either exclusively linear-cleavage peptide (especially for shorter peptides) or none at all, as was the case for the VGAG-8Aoc-FP peptide.¹³

2.2 GmPOPB

2.2.1 Structural Biology of Apo and Substrate-Bound GmPOPB.

Apo GmPOPB crystals belong to space group P212121, with one monomer in the asymmetric unit. The structure was determined at 2.4 Å resolution by molecular replacement using the β-propeller domain of the proline oligopeptidase from porcine brain (residues 82-450, PDB:1h2z) as search model. The refined apo model (PDB:5N4F) includes residues 7-222, 230-695, and 704-726, and the missing regions are presumed to be disordered portions of the protein. The protein contains two domains as observed in other POP enzymes. The domain containing the putative catalytic residues (Ser577, Asp661, His698) comprises residues 1-81 and 450-728, and the other domain is a seven bladed β-propeller, comprising residues 82-449. In the apo structure, the two domains are in an “open” conformation, in an arrangement reminiscent of a hinged lid on a bottle. This open conformation has been observed in other POPs in crystal form when free of ligand. The catalytic serine sits at the tip of a loop and points toward the β-propeller domain. The side chain oxygen of Ser577 is 5.6 Å from the side chain carboxylate of Asp661; His698 is on a loop that is disordered. Ser577 and Asp661 of GmPOPB occupy the same position as Ser554 and Asp641 in the porcine proline oligopeptidase structure. In order to obtain co-complexes, we mutated each residue of the presumed catalytic triad (mutants S577A, D661A, and H698A) to ensure inactive protein. Crystals were obtained for the 35mer complex for S577A and H698A; with the 25mer substrate S577A and D661A (Table 2). GmPOPB-S577A (the higher resolution of the pair) bound to the full-length substrate (35mer) belongs to space group P21 with four monomers in the asymmetric unit. For ease of discussion, we split the 35mer into four regions (FIG. 5b), the 10 residue leader (residues 1-10), the 8 residue core (11-18), 6 residue linker (19-24), and the 11 residue recognition tail (25-35). The refined model (PDB:5N4C) includes residues 6-225 and 228-727 of the protein and residues 3-35 of the peptide (FIG. 5c). The same inactive mutant of GmPOPB was used to obtain a complex structure with the 25mer substrate; it comprises core (residues 1-8), linker (9-14), and recognition tag (15-25). The refined model includes residues 4-727 of the protein and residues 9-25 (linker and recognition tag) of the peptide (PDB:5N4B). Although we observed residual difference electrondensity for the N-terminal residues of the 25mer, we were unable to satisfactorily model it. To observe interactions when the catalytic residue Ser577 is present, we also obtained complex structures of the H698A mutant bound to the 35mer peptide (PDB:5N4E) and the D661A mutant bound to the 25mer peptide (PDB:5N4D). In all 35mer and 25mer complexes, the enzyme adopts the same “closed” conformation in which the lid (the propeller domain) sits on top of the catalytic domain. In both 25mer complexes the N-terminal residues of the substrate (IWGIGCN) are disordered; in the S577A-35mer complex the N-terminal two residues (MF) are missing, while in the H698A complex only the first N-terminal residue is absent. There are no large differences between the protein backbone positions in the complexes with the 35mer and 25mer substrates (root mean square deviation (rmsd) of 0.48 Å over 720 Cα positions for the S577A structures).

There are also no major differences between the H698A-35mer and S577A-35mer complexes, and between the S577A-25mer and D661A-25mer complexes. Table 1 shows the data collection and refinement statistics for all structures. The recognition tail adopts an identical distorted 310 helix conformation inserted into the middle of the β-propeller domain in both the 25mer and 35mer complexes. The carboxyl terminus sits in a pocket where it makes water-mediated hydrogen bonds to the protein. To our surprise, there are only a few hydrogen bonds between the protein and the tail. Comparison to the apo structure reveals that binding of substrate induces no significant changes in the core of the propeller domain (rmsd of 0.75 Å over 360 Cα positions for the S577A structures). The changes that occur (relative to the apo structure) are in loops around the catalytic site. Significant differences between the two substrates are observed for the residues of the linker region, since it occupies distinct conformations on the 25mer and 35mer complexes. V24 faces Arg79 and V14 is toward Phe506. H23 does not form any hydrogen bonds, while H13 is hydrogen bonding with Glu601. Tyr494 is within hydrogen bonding distance from E22, while on the 25mer complex Tyr494 is interacting with W9. This peptide twisting causes a tryptophan present in the peptides from both complexes (residue 19 in 35mer, 9 in the 25mer), C-terminal to the site of cleavage, to occupy a binding pocket close to the active site. The oxyanion-stabilizing Tyr496 is in close proximity to the core proline (P8) in the 25mer structure (D661A mutant), while Tyr496 hydrogen bonds with P18 in the 35mer complex. Substrate residues I11-P18 (the core peptide is not seen in the 25mer, apart from weak density from P8 in the D661A-25mer complex) form a twisted loop, which makes contacts with the protein and within itself; atoms that will ultimately form the macrocycle are 7.6 Å apart. The core peptide interposes between substrate P18 and enzyme Ser577, which is over 8 Å away. Substrate P10, the site of proteolytic cleavage, is positioned for attack by Ser577 (the Cβ of the mutated residue is 3.3 Å from the carbonyl with plausible geometry). In the crystal structure of H698A-35mer complex, the hydroxyl of Ser577 is in hydrogen bonding distance from P10, in a position suited for nucleophilic attack but the structure is less ordered, notably the loop containing the mutation H698A.

Tyr496 is on the opposite face of the carbonyl 2.8 Å from the oxygen and positioned to stabilize the tetrahedral intermediate from attack of Ser577. Both the interaction and the role of Tyr496 are conserved in other POPs. In the 35mer complex structures, residues 2-9 adopt a helical arrangement that ends up exposed to solvent at the N-terminus and makes few contacts with the protein. In none of the GmPOPB structures that we have obtained are the catalytic residues arranged in the traditional manner, the closest approach of the His698 and Ser577 is 13 Å and residues block simple movement. To confirm the importance of the putative catalytic triad for GmPOPB, the mutants S577A, D661A, and H698A were evaluated for activity, and were inactive with both 25mer and 35mer substrates, using 5 μM GmPOPB and monitoring the reaction progress after 24 h.

2.2.2 Mutations in the Histidine Loop Decrease Enzyme Activity.

To study other residues involved in hydrolysis and macrocyclisation, additional mutants H698N, R663A, R663Q, R663K, and W695Δ (deletion) were generated. These mutants were designed based on comparison of sequence alignments between other POP enzymes and the very similar POPA enzyme from G. marginata, enzymes that solely act as proteases. Arg663 is highly conserved in POPs and thought to play a role in catalysis or substrate binding, since it makes hydrogen bonds with the peptide substrate³¹. H698N was insoluble and not evaluated. The other mutants possessed diminished activities for both peptide bond hydrolysis and macrocyclisation. The amount of macrocyclic peptide present after incubation for 16 h with the 25mer substrate was R663Q>R663A>W695Δ>R663K. When the 35mer was used as substrate, the mutants demonstrated diminished activity for peptide bond hydrolysis and almost undetectable activity for macrocyclisation. Kinetic characterization and substrate scope of GmPOPB. Previous analysis employed GmPOPB isolated from the G. marginata mushroom after transformation with Agrobacterium tumefaciens. We examined kinetic parameters and performed a substrate specificity study on the enzyme isolated employing a bacterial overexpression system. Our results on the native overexpressed enzyme confirm the previous findings obtained for protein purified from mushroom that the full-length 35mer substrate is cleaved and the resulting 25mer is released. The kinetic data for expressed protein with the 25mer but not 35mer have been previously reported. The 25mer then rebinds (in competition with the 35mer) for macrocyclisation. Cleavage and macrocyclisation do not occur in a single binding event. The 25mer accumulates as an intermediate although the proteolysis reaction is slower than macrocyclic peptide formation. Very similar values for Kₘ and k_{cat} were obtained for all full-length substrates evaluated, with Kₘ values ranging from 8 to 51 μM, while k_{cat} was between 3.2 and 35 min⁻¹. The substrate had no effect either on kinetic parameters or yield of cyclic product. Less conservative substitutions such as mutation to alanine or 9mer core (IWGIGCANP the bold underlined residue represents the insertion) led to reduced macrocyclisation and increased linear peptide, the product of peptide hydrolysis instead of macrocyclisation.

2.2.3 Equilibrium Binding of Substrates and Products.

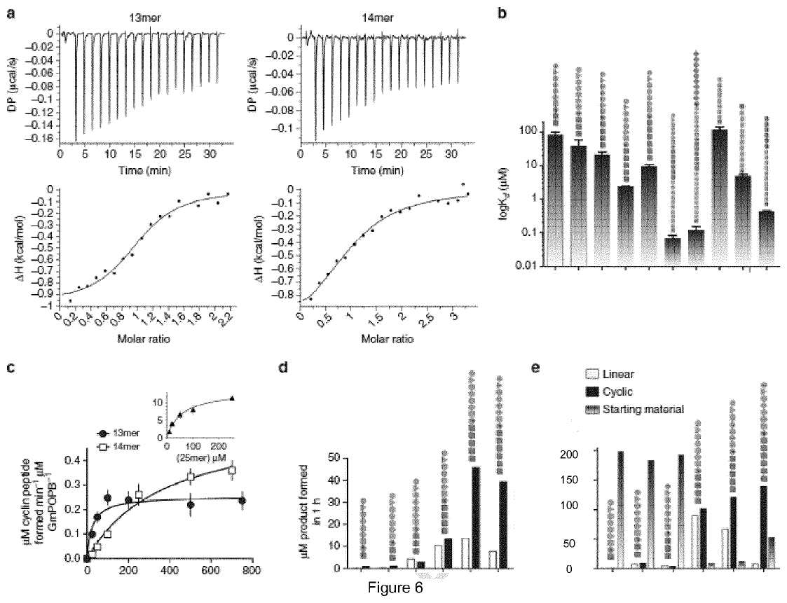

Binding of the inactive mutant S577A to the 25mer, 35mer, a series of truncated substrates (10mer-14mer), as well as the recognition sequence (17mer) WTAEHVDQTLASGNDIC, the truncated recognition sequences VDQTLASGNDIC and TLASGNDIC, and the leader peptide MFDTNATRLP were evaluated by isothermal titration calorimetry (ITC). The results of S577A with both the 25mer and recognition sequence have previously been reported. Binding of H698A to the 25mer was also measured. The only peptide showing no detectable binding at concentrations up to 1 mM was the 10-residue leader peptide MFDTNATRLP. The full-length substrates and products displayed tight binding (K_{d-25mer-S577A}=67±14 nM, K_{d-25mer-H698A}=47±11 nM, K_{d-35mer}=120±30 nM, K_{d-recognition}=430±10 nM; binding is dominated by enthalpic contributions (FIG. 6a shows representative ITC traces for the 13mer and 14mer substrates, FIG. 6b shows K_{d} values for all peptides evaluated). The inactive mutant H698A has identical K_{d}-25mer to the S577A mutant suggesting the lack of activity results from catalytic incompetence rather than disruption of substrate binding. Interestingly, despite being longer and having the potential for more interactions with the protein, the 35mer peptide shows slightly weaker binding compared to the 25mer, mostly due to decreased ΔH. A comparison of the complex structures shows that in the 35mer complex there is disorder of side chains in the segment TAEHVD (linker region) but not in the 25mer. To investigate the role of recognition tag peptides corresponding to the entire recognition sequence (linker plus tail, WTAEHVDQTLASGNDIC—17 residues), the recognition tail plus the valine from the linker (VDQTLASGNDIC 12 residues) and the highly conserved portion of the tail (TLASGNDIC 9 residues) were tested for binding and gave K_{d-17mer-recogSeq}=0.43±0.01 μM, K_{d-12mer-recogSeq}=5±1 μM and K_{d-9mer-recogSeq}=121±19 μM. Previously, we showed that the recognition sequence dominates binding, with a difference in ΔG of only 1.34 kcal mol-1 between the 17mer recognition sequence and the 25mer peptide. To explore how much contribution to the binding energy comes from the linker region, we evaluated the binding of truncated recognition sequences. Our data show that the linker region is important in binding as the loss of the linker (shrinking the recognition sequence from 17 to 12 amino acids) reduces binding affinity 20-fold. On its own, the highly conserved nine-residue tail bound rather weakly, consistent with the few interactions observed with it and the protein. Following from this finding, a series of truncated peptides (core plus parts of the linker) were tested and revealed a trend in which binding affinity increased from 10mer to 13mer (K_{d-10mer}=83±17 μM, K_{d-11mer}=39±18 μM, K_{d-12mer}=21±5 μM, K_{d-13mer}=2.4±0.1 μM) peptides, but decreased slightly with the 14mer peptide (K_{d-14mer}=9.5±1.1 μM) (FIG. 6b); the 9mer was not sufficiently soluble for analysis. We noted that the difference in affinity between the 35mer and the core plus linker (13mer) was ˜20-fold.

3. Summary

The importance of peptidic and amino acid-containing macrocycles in medicine is now clear. Robust synthetic approaches for these molecules, which can combine synthetic diversity with the ability of enzymes to catalyse challenging chemical steps, are highly desired. Here we have reported the characterization of PCY1, a macrocyclase from plant and GmPOPB a macrocyclase from fungus.

PCY1 in nature processes a wide range of substrates that vary in length and sequence, which we show can be extended to ten amino acid macrocycles. We show substrate recognition occurs in two distinct patches of the substrate, joined by a flexible linker, which underpins the promiscuity of PCY1. Based on understanding the recognition, we have demonstrated that PCY1can utilize simpler synthetically accessible substrates with a short C-terminal extension at a higher rate than other existing processes. We have also demonstrated that the enzyme can process hybrid molecules than are not solely composed of amino acids, suggesting PCY1 could represent a valuable synthetic tool.

GmPOPB processes a 35 amino-acid substrate, the longest observed for a POP. POP enzymes possess an aspartate, histidine, and serine catalytic triad. Consistent with the observation of similar binding affinities of the 25mer and 35mer (K_{d} 67 and 120 nM, respectively), the 10-residue leader (only present in 35mer) does not bind. In both the 25mer and 35mer complex structures, the recognition tail (C-terminal 11 residues) is embedded deeply into the β-propeller domain in an essentially identical arrangement. The linker region, however, adopts very different arrangements in the two complexes, thus its interactions with the protein are quite distinct in the two structures. ITC measurements show that the linker region, particularly the portion following the core peptide, makes substantial contribution to the binding energy. This is in contrast to the heterocyclase class of RIPP enzymes, where the linker plays no role and can be varied. Our data show that the structure of the linker is important in binding and determines the orientation of the substrate at the active site (thus its fate). Previous kinetic assays and ours reveal that after removal of the leader, the remaining 25mer is released from GmPOPB, then it rebinds and undergoes the macrocyclisation reaction. In the 35mer complex, the core and linker adopt a tightly packed arrangement that is wedged between the active site loops. We conclude that the linker and/or core are unable to refold to the conformational arrangement seen in the 25mer complex (required for macrocyclisation) in situ on a timescale comparable to dissociation. Having identified the key role of the linker, we predicted that it should be possible to design simpler macrocyclisation substrates that lacked the recognition tail. This would be valuable since the use of 25mer substrates to make eight residue macrocycles is not economic. ITC shows a 10-fold reduction in binding from 35mer to 13mer, kinetic analysis reveals the 13mer substrate has a Kₘ (25 μM) within error to the 25mer substrate (50 μM), while the 14mer possesses higher Kₘ (380 μM). Similar kcat values were observed with both shorter substrates (0.49 and 0.58 min⁻¹ for the 13mer and 14mer, respectively, FIG. 6c) but these are smaller than the 25mer (18 min⁻¹). Linear peptide (product of hydrolysis instead of macrocyclisation) was observed when shorter peptides were utilized as substrates (FIG. 6d) consistent with the linker playing a key role in substrate positioning. After 16 h of reaction both 13mer and 14mer substrates produce similar amounts of macrocycle, but the 14mer generates less linear product. Linear peptide produced this may not be a significant drawback, as purification of macrocycles from liner peptides is straightforward.

Compared to PatG this represents a significant improvement, since biocatalytic reactions with PatG in vitro can require over 7 days and utilize up to stoichiometric amounts enzyme. GmPOPB is an unusual enzyme catalyzing, depending on substrate length, proteolysis, or macrocyclisation using the same catalytic machinery. Previous work had identified the crucial nature of the recognition sequence in the substrate, but suggested its full length was a requirement for macrocyclisation. Our structural work supported by calorimetry and kinetics reveals that shorter peptides are suitable substrates. GmPOPB recognizes residues within the linker connecting the core and the recognition tail, and this recognition is critical to position the substrate for macrocyclisation. A substrate with five or six C-terminal residues (as opposed to 17) chosen to mimic the linker can be efficiently macrocyclized at synthetically useful rates.

This work highlights the power of structural and mechanistic studies to redesign substrates or enzymes for use in biotechnology.

TABLE 1

| Substrate | Kᵢₙ (μM) | kₒᵤₜ (s⁻¹) | kₒᵤₜ/Kᵢₙ (M) | Method |
|---|---|---|---|---|
| PresegB1 | 0.45 ± 0.17 | 0.37 ± 0.02 | 822.22 ± 313.78 | LC/MS (UV_{280 nm}) |
| PresegB1 | 0.25 ± 0.01 | 0.14 ± 0.01 | 560.00 ± 45.85 | Single injection kinetic ITC |
| PresegF1 | 2.40 ± 0.51 | 0.12 ± 0.01 | 50.00 ± 11.41 | LC/MS (ion counts) |
| PresegF1- | 227.4 ± 15.7 | 0.0052 ± 0.0001 | 22.75 ± 1.69 | LC/MS (ion counts) |
| truncated | | | | |

TABLE 2

| | GmPOPB- | | GmPOPB- | | GmPOPB- | | GmPOPB- | | | |
|---|---|---|---|---|---|---|---|---|---|---|
| | S577A_25mer | | D661A_25mer | | S577A_35mer | | H698A_35mer | | apo_GmPOPB | |
| Data collection | | | | | | | | | | |
| Space group | P 2₁ 2₁ 2₁ | | P 2₁ 2₁ 2₁ | | P 2₁ | | P 2₁ 2₁ 2₁ | | P 2₁ 2₁ 2₁ | |
| Cell dimensions | | | | | | | | | | |
| a, b, c | 99.1, 114.7, 141.3 | | 99.0, 114.9, 141.3 | | 100.8, 142.6, 116.4 | | 99.2, 115 141.5 | | 90.89, 105.62, 86.9 | |
| α, β, γ | 90.0, 90.0, 90.0 | | 90.0, 90.0, 90.0 | | 90.0, 90.29, 90.0 | | 90.0, 90.0, 90.0 | | 90.0, 90.0, 90.0 | |
| Resolution | 1.44 | (1.49-1.44) | 1.62 | (1.678-1.62) | 2.19 | (2.268-2.19) | 2.9 | (3.0-2.9) | 2.4 | (2.49-2.4) |
| Unique reflections | 288,485 | (28,320) | 204,059 | (20,188) | 163,036 | (16,107) | 32,664 | (1690) | 31,441 | (2534) |
| Completeness (%) | 99.7 | (99) | 100 | (100) | 97 | (96) | 97 | (93) | 99 | (77) |
| Mean I/sigma (I) | 9.3 | (1.3) | 6.9 | (1.0) | 6.7 | (1.2) | 6.8 | (1.7) | 14.5 | (2.2) |
| R-merge | 0.114 | (1.01) | 0.059 | (0.69) | 0.112 | (1.0) | 0.068 | (0.53) | 0.029 | (0.56) |
| R-meas | 0.063 | (0.588) | 0.084 | (0.98) | 0.27 | (1.5) | 0.096 | (0.52) | 0.036 | (0.72) |
| Redundancy | 6.0 | (6.6) | 6.7 | (6.6) | 2.0 | (2.0) | 1.8 | (1.6) | 5.1 | (3.3) |
| Refinement | | | | | | | | | | |
| Resolution range | 43.3-1.44 | | 46.9-1.62 | | 82.3-2.19 | | 32.0-2.9 | | 45.44-2.4 | |
| Reflections used in refinement | 288,428 | (28,313) | 203,960 | (20,185) | 163,135 | (16,107) | 32,664 | (1690) | 31,437 | (2534) |

| | | | | | |
|---|---|---|---|---|---|
| R_{work}/R_{free} | 0.17/0.19 | 0.20/0.23 | 0.23/0.26 | 0.255/0.303 | 0.21/0.25 |
| Protein residues | 1478 | 1478 | 3014 | 1484 | 703 |
| RMS(bonds) | 0.015 | 0.020 | 0.012 | 0.007 | 0.007 |
| RMS(angles) | 1.28 | 1.82 | 1.42 | 1.10 | 1.17 |
| Ramachandran favored (%) | 97 | 97 | 97 | 97 | 97 |
| Ramachandran allowed (%) | 2.5 | 2.5 | 3.1 | 2.9 | 2.3 |
| Ramachandran outliers (%) | 0.14 | 0.3 | 0.3 | 0.48 | 0.29 |
| Rotamer outliers (%) | 0.24 | 1.4 | 0.95 | 1.1 | 0.34 |
| Clashscore | 3.73 | 1.63 | 1.02 | 3.42 | 2.64 |
| Average B-factor | 15 | 21 | 15 | 32 | 44 |
| Macromolecules | 14 | 20 | 14 | 34 | 43 |
| Solvent | 27 | 31 | 43 | 37 | 38 |

Sequences
PCY1 enzyme - (724 aa) (S. vaccaria)
SEQ ID NO: 1

| | | | | |
|---|---|---|---|---|
| 001 | matsgfskpl | hyppvrrdet | vvddyfgvkv | adpyrwledp |
| | nseetkefvd | nqeklansvl | | |
| 061 | eecelidkfk | qkiidfvnfp | rcgvpfrran | kyfhfynsgl |
| | qaqnvfqmqd | dldgkpevly | | |
| 121 | dpnlreggrs | glslysysed | akyfafgihs | gltewvtiki |
| | lktedrsylp | dtlewvkfsp | | |
| 181 | aiwthdnkgf | fycpypplke | gedhmtrsav | nqearyhflg |
| | tdqsedillw | rdlenpahhl | | |
| 241 | kcqitddgky | fllyildgcd | dankvycldl | tklpnglesf |
| | rgredsapfm | klidsfdasy | | |
| 301 | taiandgsvf | tfqtnkdapr | kklvrvdlnn | psvwtd1vpe |
| | skkdllesah | avnenqlilr | | |
| 361 | ylsdvkhvle | irdlesgalq | hrlpidigsv | dgitarrrds |
| | vvffkftsil | tpgivyqcdl | | |
| 421 | kndptqlkif | resvvpdfdr | sefevkqvfv | pskdgtkipi |
| | fiaarkgisl | dgshpcemhg | | |
| 481 | yggfginmmp | tfsasrivfl | khlggvfcla | nirgggeyge |
| | ewhkagfrdk | kqnvfddfis | | |
| 541 | aaeylissgy | tkarrvaieg | gsnggllvaa | cinqrpdlfg |
| | caeancgvmd | mlrfhkftlg | | |
| 601 | ylwtgdygcs | dkeeefkwli | kyspihnvrr | pweqpgneet |
| | qypatmilta | dhddrvvplh | | |
| 661 | sfkllatmqh | vlctsledsp | qknpiiariq | rkaahygrat |
| | mtgiaevadr | ygfmakalea | | |
| 721 | pwid | | | |

GmPOPB
SEQ ID NO: 2

| | | | | |
|---|---|---|---|---|
| 001 | mssvtwapgn | ypstrrsdhv | dtyqsaskge | vpvpdpyqwl |
| | eestdevdkw | ttaqadlaqs | | |
| 061 | yldqnadiqk | laekfrasrn | yakfsaptll | ddghwywfyn |
| | rglqsqsvly | rskepalpdf | | |
| 121 | skgddnvgdv | ffdpnvlaad | gsagmvlckf | spdgkffaya |
| | vshlggdyst | iyvrstsspl | | |
| 181 | sqasvaggvd | grlsdevkwf | kfstiiwtkd | skgflyqryp |
| | arerhegtrs | drnammcyhk | | |
| 241 | vgttgeedii | vyqdnehpew | iygadtsedg | kylylyqfkd |
| | tskknllwva | eldedgvksg | | |
| 301 | ihwrkvvney | aadyniitnh | gslvyiktnl | napqykviti |
| | dlskdepeir | dfipeekdak | | |
| 361 | laqvncanee | yfvaiykrnv | kdeiylyska | gvqltrlapd |
| | fvgaasianr | qkqthffltl | | |
| 421 | sgfntpgtia | rydftapetq | rfsilrttkv | neldpddfes |
| | tqvwyeskdg | tkipmfivrh | | |
| 481 | kstkfdgtaa | aiqygyggfa | tsadpffspi | iltflqtyga |
| | ifavpsirgg | gefgeewhkg | | |
| 541 | grretkvntf | ddfiaaaqfl | vknkyaapgk | vaingasngg |
| | llvmgsivra | pegtfgaavp | | |
| 601 | eggvadllkf | hkftggqawi | seygnpsipe | efdyiyplsp |
| | vhnvrtdkvm | patlitvnig | | |
| 661 | dgrvvpmhsf | kfiatlqhnv | pqnphpllik | idkswlghgm |
| | gkptdknvkd | aadkwgfiar | | |
| 721 | alglelktve | | | |

### REFERENCES

1. Driggers, E. M. et al. Nat Rev Drug Discov 2008, 7 (7), 608-24.

2. Lipinski, C. A.; Lombardo, F.; Dominy, B. W.; Feeney, P. J., Experimental and computational approaches to estimate solubility and permeability in drug discovery and development settings. Adv Drug Deliver Rev 1997, 23 (1-3), 3-25.

3. Terrett, N., Drugs in middle space. MedChemComm 2013, 4 (3), 474-475.

4. Zorzi, A.; Deyle, K.; Heinis, C., Cyclic peptide therapeutics: past, present and future. Curr Opin Chem Biol 2017, 38, 24-29.

5. Yu, X.; Sun, D., Macrocyclic drugs and synthetic methodologies toward macrocycles. Molecules 2013, 18 (6), 6230-68.

6. Marti-Centelles, V.; Pandey, M. D.; Burguete, M. I.; Luis, S. V., Macrocyclisation Reactions: The Importance of Conformational, Configurational, and Template-Induced Preorganization. Chem Rev 2015, 115 (16), 8736-834.

7. Sardar, D.; Lin, Z.; Schmidt, E. W., Modularity of RiPP Enzymes Enables Designed Synthesis of Decorated Peptides. Chem Biol 2015, 22 (7), 907-16.

8. Sivonen, K.; Leikoski, N.; Fewer, D. P.; Jokela, J., Cyanobactins-ribosomal cyclic peptides produced by cyanobacteria. Appl Microbiol Biot 2010, 86 (5), 1213-1225.

9. Sardar, D.; Tianero, M. D.; Schmidt, E. W., Directing Biosynthesis: Practical Supply of Natural and Unnatural Cyanobactins. Methods Enzymol 2016, 575, 1-20.

10. Tianero, M. D.; Pierce, E.; Raghuraman, S.; Sardar, D.; McIntosh, J. A.; Heemstra, J. R.; Schonrock, Z.; Covington, B. C.; Maschek, J. A.; Cox, J. E.; Bachmann, B. O.; Olivera, B. M.; Ruffner, D. E.; Schmidt, E. W., Metabolic model for diversity-generating biosynthesis. Proc Nati Aced Sci USA 2016, 113 (7), 1772-7.

11. Oueis, E.; Adamson, C.; Mann, G.; Ludewig, H.; Redpath, P.; Migaud, M.; Westwood, N. J.; Naismith, J. H., Derivatisable Cyanobactin Analogues: A Semisynthetic Approach. Chembiochem 2015, 16 (18), 2646-50.

12. Oueis, E.; Jaspars, M.; Westwood, N. J.; Naismith, J. H., Enzymatic Macrocyclisation of 1,2,3-Triazole Peptide Mimetics. Angew Chem Weinheim Bergstr Ger 2016, 128 (19), 5936-5939.

13. Oueis, E.; Nardone, B.; Jaspars, M.; Westwood, N. J.; Naismith, J. H., Synthesis of Hybrid cyclopeptides through Enzymatic Macrocyclisation. ChemistryOpen 2017, 6 (1), 11-14.

14. Houssen, W. E.; Bent, A. F.; McEwan, A. R.; Pieiller, N.; Tabudravu, J.; Koehnke, J.; Mann, G.; Adaba, R. I.; Thomas, L.; Hawes, U. W.; Liu, H.; Schwarz-Linek, U.; Smith, M. C.; Naismith, J. H.; Jaspars, M., An efficient method for the in vitro production of azol(in)e-based cyclic peptides. Angew Chem Int Ed Engl 2014, 53 (51), 14171-4.

15. McIntosh, J. A.; Robertson, C. R.; Agarwal, V.; Nair, S. K.; Bulaj, G. W.; Schmidt, E. W., Circular logic: nonribosomal peptide-like macrocyclisation with a ribosomal peptide catalyst. J Am Chem Soc 2010, 132 (44), 15499-501.

16. Luo, H.; Hong, S. Y.; Sgambelluri, R. M.; Angelos, E.; Li, X.; Walton, J. D., Peptide macrocyclisation catalysed by a prolyl oligopeptidase involved in alphaamanitin biosynthesis. Chem Biol 2014, 21 (12), 1610-7.29

17. Barber, C. J.; Pujara, P. T.; Reed, D. W.; Chiwocha, S.; Zhang, H.; Covello, P. S., The two-step biosynthesis of cyclic peptides from linear precursors in a member of the plant family Caryophyllaceae involves cyclization by a serine protease-like enzyme. J Biol Chem 2013, 288 (18), 12500-10.

18. Nguyen, G. K.; Wang, S.; Qiu, Y.; Hemu, X.; Lian, Y.; Tam, J. P., Butelase 1 is an Asx-specific ligase enabling peptide macrocyclisation and synthesis. Nat Chem Biol 2014, 10 (9), 732-8.

19. Nguyen, G. K.; Kam, A.; Loo, S.; Jansson, A. E.; Pan, L. X.; Tam, J. P., Butelase 1: A Versatile Ligase for Peptide and Protein Macrocyclisation. J Am Chem Soc 2015, 137 (49), 15398-401.

20. Nguyen, G. K.; Qiu, Y.; Cao, Y.; Hemu, X.; Liu, C. F.; Tam, J. P., Butelasemediated cyclization and ligation of peptides and proteins. Nat Protoc 2016, 11 (10), 1977-1988.

21. Li, K.; Condurso, H. L.; Li, G.; Ding, Y.; Bruner, S. D., Structural basis for precursor protein-directed ribosomal peptide macrocyclisation. Nat Chem Biol 2016, 12 (11), 973-979.

22. Yang, R.; Wong, Y. H.; Nguyen, G. K. T.; Tam, J. P.; Lescar, J.; Wu, B., Engineering a Catalytically Efficient Recombinant Protein Ligase. J Am Chem Soc 2017, 139 (15), 5351-5358.

23. Czekster, C. M.; Ludewig, H.; McMahon, S. A.; Naismith, J. H., Characterization of a dual function macrocyclase enables design and use of efficient macrocyclisation substrates. Nature Communications 2017, (manuscript under revision since February 2017).

24. Czekster, C. M.; Naismith, J. H., Kinetic landscape of a peptide bond-forming prolyl oligopeptidase. Biochemistry 2017, 56 (15), 2086-2095.

25. Arnison, P. G.; et al Nat Prod Rep 2013, 30 (1), 108-60.

26. U.S. Pat. No. 9,394,561B

1. A method of producing a macrocyclic molecule comprising;
(i) providing a linear precursor peptide consisting of a core region and a cyclisation tag of 10 or fewer amino acid residues; and,
(ii) reacting the precursor peptide with an isolated prolyl oligopeptidase (POP) macrocyclase to produce a macrocyclic molecule having the core region.

2. A method according to claim 1 wherein the core region is located at the N terminus of the precursor peptide and is directly linked via a peptidyl bond at its C terminal end to the cyclisation tag.

3. A method according to claim 1 or claim 2 wherein the core region consists of 4 to 30 residues.

4. A method according to any one of the preceding claims wherein the core region comprises one or more non-peptidyl linkages.

5. A method according to any one of the preceding claims wherein the cyclisation tag is located at the C terminus of the precursor peptide and is linked directly to the core region through a peptidyl bond.

6. A method according to any one of the preceding claims wherein the cyclisation tag consists of 3-10 amino acid

7. A method according to any one of the preceding claims wherein the precursor peptide is reacted with the POP macrocyclase in the presence of a catalytic peptide.

8. A method according to any one of the preceding claims wherein the catalytic peptide consists of 6-8 residues from the C terminus of the wild type substrate of the POP macrocyclase.

9. A method according to any one of the preceding claims wherein the POP macrocyclase is a plant POP macrocyclase.

10. A method according to claim 9 wherein the POP macrocyclase is an S vaccaria PCY1 macrocyclase

11. A method according to claim 9 or claim 10 wherein the POP macrocyclase comprises an amino acid sequence having at least 60% sequence to SEQ ID NO: 1.

12. A method according to any one of claims 9 to 11 wherein the core region consists of 5-10 residues

13. A method according to any one of claims 9 to 12 wherein the cyclisation tag consists of 3-6 residues

14. A method according to any one of claims 9 to 13 wherein the cyclisation tag consists of IQTQVS, IQTQV, IQ, IQTQ, IQT, IQD; AKDAEN, AKDAE, AKDA, AKD; FQAKDV, FQAKD, FQAK, FQA or FQ or variants thereof

15. A method according to any one of claims 9 to 13 wherein the catalytic peptide consists of the sequence (R/D/E)NAS(A/S)PV or (R/D/E)AS(A/S)PV.

16. A method according to any one of claims 1 to 8 wherein the POP macrocyclase is a fungal POP macrocyclase.

17. A method according to claim 16 wherein the POP macrocyclase is a Galerina marginata GmPOPB macrocyclase.

18. A method according to claim 17 wherein the POP macrocyclase comprises an amino acid sequence having at least 60% sequence to SEQ ID NO: 2.

19. A method according to claim 17 or claim 18 wherein the core region consists of 5-10 residues.

20. A method according to any one of claims 17 to 19 wherein the core region consists of IWGIGC(N/D)P or a variant thereof.

21. A method according to any one of claims 17 to 20 wherein the cyclisation tag consists of 3-6 residues

22. A method according to any one of claims 17 to 21 wherein the cyclisation tag consists of WTAEH or WTAEHV or a variant thereof

23. A method according to any one of claims 17 to 22 wherein the catalytic peptide consists of the sequence ASGNDIC.

24. A method according to any one of claims 1 to 23 wherein the precursor peptide is immobilised and the POP macrocyclase is free in solution.

25. A method according to any one of claims 1 to 23 wherein the POP macrocyclase is immobilised and the precursor peptide is free in solution.

26. A method according to any one of claims 1 to 25 wherein the macrocyclic molecule is a macrocyclic peptide.

27. A method according to claim 26 wherein the macrocyclic peptide is an amatoxin, phallotoxin, cyclotide or cyanobactin.

28. A method according to any one of claims 1 to 27 wherein the macrocyclic molecule is subjected to further chemical modification.

29. A method according to claim 28 wherein the macrocyclic molecule is subjected to oxidation, hydroxylation, epimerisation, cross-linking and/or prenylation

30. A method according to any one of claims 1 to 29 wherein the macrocyclic molecule is labeled with a detectable label.

31. A method according to any one of claims 1 to 30 comprising isolating and/or purifying the macrocyclic molecule

32. A precursor peptide comprising a core region and a cyclisation tag of 10 or fewer amino acid residues.

33. A library of precursor peptides comprising a core region and a cyclisation tag of 10 or fewer amino acid residues, wherein the library comprises a diverse core region and the same cyclisation tag.

34. A library according to claim 33 wherein the precursor peptides are immobilised on beads.

35. A method of screening a macrocyclic peptide library comprising;
(i) providing a population of precursor peptides, each precursor consisting of a core region and a cyclisation tag having 10 or fewer amino acids, wherein the core region is diverse in the population and the cyclisation tag is the same,
(ii) treating said population with a prolyl oligopeptidase (POP) macrocyclase to convert the core region into a macrocyclic molecule,
(iii) screening the macrocyclic molecules for activity,
(iv) identifying an active macrocyclic molecule.

36. A method of screening a macrocyclic peptide library comprising;
(i) providing a diverse population of core regions attached to beads, each bead having a first and a second copy of the core regions attached thereto, wherein the first copy but not the second copy is attached to the bead via a cyclisation tag having 10 or fewer amino acids,
(ii) treating said beads with a prolyl oligopeptidase (POP) macrocyclase to convert the first copy of the core region into a macrocyclic molecule and release the macrocyclic molecules from the beads,
(iii) screening the macrocyclic molecules for activity,
(iv) identifying an active macrocyclic molecule
(v) identifying the bead from which the macrocyclic molecule was released, and
(vi) sequencing the second copy of the core region attached to the bead.

37. A kit for use in producing a macrocyclic molecule comprising;
(i) a precursor peptide comprising a core region and a cyclisation tag of 10 or fewer amino acid residues, and
(ii) an isolated prolyl oligopeptidase (POP) macrocyclase.

38. A kit according to claim 37 further comprising a catalytic peptide.

39. A kit according to claim 37 or 38 further comprising a solid support.
